(19) United States
(12) Patent Application Publication
(10) Pub. No.: US 2026/0357334 A1
(43) Pub. Date: Nov. 12, 2026

(54) METHODS AND SYSTEMS FOR IDENTIFYING COPY NUMBER SIGNATURES

(71) Applicant: Foundation Medicine, Inc., Boston, MA (US)

(72) Inventors: Jay MOORE, New York, NY (US); Dexter X. JIN, Stoneham, MA (US); Ethan S. SOKOL, Somerville, MA (US); Justin NEWBERG, Cambridge, MA (US)

(73) Assignee: Foundation Medicine, Inc., Boston, MA (US)

(21) Appl. No.: 19/117,621

(22) PCT Filed: Oct. 4, 2023
(86) PCT No.: PCT/US2023/075910

§ 371 Date: Apr. 1, 2025

Related U.S. Application Data
(60) Provisional application No. 63/413,559, filed on Oct. 5, 2022.

(51) Int. Cl. C12Q 1/6869; A61K 31/502; A61K 31/55; C12Q 1/6827; C12Q 1/6855; C12Q 1/6886; G16B 20/10; G16B 20/20; G16B 30/00; G16B 40/20

(52) U.S. Cl. CPC C12Q 1/6869; A61K 31/502; A61K 31/55; C12Q 1/6827; C12Q 1/6855; C12Q 1/6886; G16B 20/10; G16B 20/20; G16B 30/00; G16B 40/20; C12Q 2600/106; C12Q 2600/156; G01N 2800/52; G01N 2800/7028

(57) ABSTRACT

Methods and systems for calling copy number signatures based on nucleic acid sequencing data are described. The methods may comprise, for example, determining a copy number profile for an individual based on sequence read data; determining a copy number feature value for each of a plurality of copy number features; analyzing the copy number feature values using a first trained model to determine a probability that one or more copy number feature subgroups are represented in the copy number profile; analyzing the determined probabilities using a second trained model to identify one or more copy number signatures and their corresponding copy number signature scores; comparing the copy number signature scores to corresponding predetermined thresholds; and identifying, based on the comparison, a dominant copy number signature or a set of one or more copy number signatures that are components of the copy number profile for the individual.

### CROSS-REFERENCE TO RELATED APPLICATIONS

This application claims the priority benefit of U.S. Provisional Patent Application Ser. No. 63/413,559, filed Oct. 5, 2022, the contents of which are incorporated herein by reference in their entirety.

### FIELD OF THE INVENTION

The present disclosure relates generally to methods and systems for analyzing genomic profiling data, and more specifically to methods and systems for identifying copy number signatures in samples from individuals using genomic profiling data.

### BACKGROUND

Copy number signatures—patterns of copy number alterations accumulated across a genome that, in turn, are based on patterns of copy number features present in the genome—have potential use for stratification of disease (e.g., cancer), and as biomarkers for disease diagnosis, prognosis, and prediction of treatment outcomes. However, there can be multiple mutational processes that drive copy-number alteration in human cancers that are not readily distinguished based on sequence data alone (see, e.g., Macintyre, et al. (2018), “Copy-Number Signatures and Mutational Processes in Ovarian Carcinoma”, Nature Genetics 50: 1262-1270), and which therefore present a challenge both for identifying a dominant mutational process in a given individual tumor sample and for the development of targeted therapies for cancers in which genomic alterations are dominated by copy-number alterations. Thus, there is a need for novel approaches to distinguish between coexistent copy number signatures, and to identify a dominant copy number signature, in tumor samples from individuals (e.g., patients).

### BRIEF SUMMARY OF THE INVENTION

Disclosed herein are methods and systems for identifying copy number signature(s) in a copy number profile for a sample from an individual (e.g., a patient), and for identifying the dominant copy number signature, or a set of one or more copy number signatures that constitute significant components of the copy number profile for the individual. Advantages of the disclosed methods and systems over previous work include utilization of off-target sequence read data derived from a targeted sequencing assay to maximize coverage of copy number features while minimizing sequencing costs, and the identification of an expanded set of copy number signatures as compared to those described in previous work.

Disclosed herein are methods comprising: providing a plurality of nucleic acid molecules obtained from a sample from an individual diagnosed with or suspected of having a disease; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, targeted sequence read data for the sample derived from the individual; determining, using the one or more processors, a copy number profile having a plurality of copy number features for the individual based on the targeted sequence read data for the sample; determining, using the one or more processors, a copy number feature value for each of the plurality of copy number features; performing, using the one or more processors, an analysis of the copy number feature values for each of a plurality of copy number features using a first trained model to determine a probability that each of one or more copy number feature subgroups is represented in the copy number profile for the sample; performing, using the one or more processors, an analysis of the probabilities determined for each of the one or more copy number feature subgroups using a second trained model to identify one or more copy number signatures represented in the copy number profile for the individual, and to determine one or more corresponding copy number signature scores; comparing, using the one or more processors, the copy number signature scores for the one or more identified copy number signatures to one or more corresponding predetermined thresholds; and identifying, based on the comparison, a dominant copy number signature for the individual; or identifying, based on the comparison, one or more copy number signatures that are components of the copy number profile for the individual.

In some embodiments, the method further comprises confirming a diagnosis of the disease for the individual based on the dominant copy number signature or the one or more copy number signatures identified for the individual. In some embodiments, the method further comprises predicting a treatment outcome for the individual based on the dominant copy number signature or the one or more copy number signatures identified for the individual. In some embodiments, the method further comprises selecting a treatment for the individual based on the dominant copy number signature or the one or more copy number signatures identified for the individual. In some embodiments, the method further comprises adjusting a treatment dosage for the individual based on the dominant copy number signature or the one or more copy number signatures identified for the individual. In some embodiments, the method further comprises selecting the individual for inclusion in a clinical trial based on the dominant copy number signature or the one or more copy number signatures identified for the individual. In some embodiments, the method further comprises identifying the individual as a candidate for chemotherapy, radiation therapy, or surgery based on the dominant copy number signature or the one or more copy number signatures identified for the individual. In some embodiments, the method further comprises identifying the individual as a candidate for hospice care based on the dominant copy number signature or the one or more copy number signatures identified for the individual.

In some embodiments, the copy number profile for the individual is determined based on single nucleotide polymorphisms (SNPs) located in the targeted sequence read data for the sample. In some embodiments, the single nucleotide polymorphisms (SNPs) are located in on-target sequence read data. In some embodiments, the copy number profile for the individual is determined based on off-target sequence reads in the targeted sequence read data for the sample.

In some embodiments, the copy number profile for the individual is determined based on a combination of on-target and off-target sequence reads in the targeted sequence read data for the sample.

In some embodiments, the analysis to identify one or more copy number signatures represented in the copy number profile of the individual comprises an analysis of both the probabilities determined for each of the one or more copy number feature subgroups and a presence of one or more genomic features detected in the targeted sequence read data for the individual using the second trained model. In some embodiments, the one or more genomic features comprise one or more short variants, one or more indels, one or more biallelic loss-of-function variants, one or more rearrangements, or any combination thereof.

In some embodiments, the subject is suspected of having or is determined to have cancer.

In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the method further comprises treating the subject with an anti-cancer therapy. In some embodiments, the anti-cancer therapy comprises a targeted anti-cancer therapy. In some embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer.

In some embodiments, one or more of the plurality of sequencing reads overlap one or more gene loci within one or more subgenomic intervals in the sample. In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In some embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-10, IL-6, IL-6R, JAK1, JAK2, JAK3, KIT, KRAS, MEK, MET, MSI-H, mTOR, PARP, PD-1, PDGFR, PDGFRa, PDGFRP, PD-L1, PI3K6, PIGF, PTCH, RAF, RANKL, RET, ROS1, SLAMF7, VEGF, VEGFA, VEGFB, or any combination thereof.

In some embodiments, the method further comprises generating, by the one or more processors, a report indicating the dominant copy number signature or the one or more copy number signature components identified for the individual. In some embodiments, the method further comprises transmitting the report to a healthcare provider. In some embodiments, the report is transmitted via a computer network or a peer-to-peer connection.

Also disclosed herein are methods, comprising: receiving, at one or more processors, targeted sequence read data for a sample derived from an individual diagnosed with or suspected of having a disease; determining, using the one or more processors, a copy number profile having a plurality of copy number features for the individual based on the targeted sequence read data for the sample; determining, using the one or more processors, a copy number feature value for each of the plurality of copy number features; performing, using the one or more processors, an analysis of the copy number feature values for each of a plurality of copy number features using a first trained model to determine a probability that each of one or more copy number feature subgroups is represented in the copy number profile for the sample; performing, using the one or more processors, an analysis of the probabilities determined for each of the one or more copy number feature subgroups using a second trained model to identify one or more copy number signatures represented in the copy number profile for the individual, and to determine one or more corresponding copy number signature scores; comparing, using the one or more processors, the copy number signature scores for the one or more identified copy number signatures to one or more corresponding predetermined thresholds; and identifying, based on the comparison, a dominant copy number signature for the individual; or identifying, based on the comparison, one or more copy number signatures that are components of the copy number profile for the individual.

In some embodiments, the method further comprises confirming a diagnosis of the disease for the individual based on the dominant copy number signature or the one or more copy number signatures identified for the individual. In some embodiments, the method further comprises predicting a treatment outcome for the individual based on the dominant copy number signature or the one or more copy number signatures identified for the individual. In some embodiments, the method further comprises selecting a treatment for the individual based on the dominant copy number signature or the one or more copy number signatures identified for the individual. In some embodiments, the method further comprises adjusting a treatment dosage for the individual based on the dominant copy number signature or the one or more copy number signatures identified for the individual. In some embodiments, the method further comprises selecting the individual for inclusion in a clinical trial based on the dominant copy number signature or the one or more copy number signatures identified for the individual. In some embodiments, the method further comprises identifying the individual as a candidate for chemotherapy, radiation therapy, or surgery based on the dominant copy number signature or the one or more copy number signatures identified for the individual. In some embodiments, the method further comprises identifying the individual as a candidate for hospice care based on the dominant copy number signature or the one or more copy number signatures identified for the individual.

In some embodiments, the copy number profile for the individual is determined based on single nucleotide polymorphisms (SNPs) located in the targeted sequence read data for the sample. In some embodiments, the single nucleotide polymorphisms (SNPs) are located in on-target sequence read data. In some embodiments, the copy number profile for the individual is determined based on off-target sequence reads in the targeted sequence read data for the sample.

In some embodiments, the copy number profile for the individual is determined based on a combination of on-target and off-target sequence reads in the targeted sequence read data for the sample. In some embodiments, the off-target sequence reads comprise an average sequencing coverage of less than 1.0×, 0.9×, 0.8×, 0.7×, 0.6×, 0.5×, 0.4×, 0.3×, 0.2×, or 0.1×.

In some embodiments, the analysis to identify one or more copy number signatures represented in the copy number profile of the individual comprises an analysis of both the probabilities determined for each of the one or more copy number feature subgroups and a presence of one or more genomic features detected in the targeted sequence read data for the individual using the second trained model. In some embodiments, the one or more genomic features comprise one or more short variants, one or more indels, one or more biallelic loss-of-function variants, one or more rearrangements, or any combination thereof.

In some embodiments, the plurality of copy number features comprises a number of breakpoints per 10 Mb of genomic sequence, a number of breakpoints per 25 Mb of genomic sequence, a number of breakpoints per 50 Mb of genomic sequence, a number of breakpoints per 100 Mb of genomic sequence, a number of breakpoints per chromosome arm for each chromosome, a magnitude of a copy number change between any two adjacent copy number segments; a length of each copy number segment; the copy number of segments; a count of contiguous oscillating copy number chains, or any combination thereof.

In some embodiments, the first trained model comprises a trained statistical model. In some embodiments, the first trained model comprises a trained machine learning model. In some embodiments, the first trained model is updated based on the one or more copy number signatures identified in the copy number profile of the individual. In some embodiments, the first trained model comprises a latent class analysis model or a mixture model. In some embodiments, the first trained model is trained on copy number feature values calculated for each of the plurality of copy number features based on copy number profiles for a cohort of individuals diagnosed with the disease.

In some embodiments, the disease comprises a cancer. In some embodiments, the cancer comprises ovarian cancer, prostate cancer, or colorectal cancer.

In some embodiments, the second trained model comprises a non-negative matrix factorization (NMF) model or a machine learning model. In some embodiments, the second trained model comprises a machine learning model, and the machine learning model comprises a supervised, semi-supervised, or unsupervised machine learning model. In some embodiments, the second trained model comprises a supervised machine learning model, and the supervised machine learning model comprises an XGBoost model.

In some embodiments, the second trained model is trained on copy number feature subgroups identified in data for a cohort of individuals diagnosed with the disease. In some embodiments, the second trained model is trained on copy number feature subgroups identified in data for a cohort of individuals comprising individuals diagnosed with a plurality of different diseases. In some embodiments, the plurality of different diseases comprises a plurality of different cancers. In some embodiments, the plurality of different cancers comprises ovarian cancer, breast cancer, non-small cell lung carcinoma (NSCLC), esophageal cancer, prostate cancer, pancreatic cancer, colorectal cancer, or any combination thereof.

In some embodiments, the second trained model is trained to identify copy number signatures based on the probabilities determined for each of the one or more copy number feature subgroups and based on the presence of one or more additional genomic features identified in the targeted sequence read data. In some embodiments, the one or more additional genomic features comprise one or more short variants, one or more indels, one or more biallelic loss-of-function variants, one or more rearrangements, or any combination thereof.

In some embodiments, the one or more corresponding predetermined thresholds are determined based on an analysis of copy number signature scores for a cohort of individuals diagnosed with the disease. In some embodiments, the one or more corresponding predetermined thresholds are determined based on an analysis of copy number signature scores for a cohort of individuals comprising individuals diagnosed with a plurality of different diseases. In some embodiments, the one or more corresponding predetermined thresholds are determined based on an analysis of data for a cohort of individuals that comprises both copy number signature scores and clinical data. In some embodiments, the clinical data comprises the individual's sex, age, gender, height, weight, clinical history, family history, sample type, tumor stage, tumor grade, or any combination thereof.

In some embodiments, the identification of a dominant copy number signature or of one or more copy number signature components is used to diagnose or confirm a diagnosis of disease in the individual. In some embodiments, the disease is cancer. In some embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the method further comprises selecting an anti-cancer therapy to administer to the subject based on the identification of the dominant copy number signature or of the one or more copy number signature components. In some embodiments, the method further comprises determining an effective amount of an anti-cancer therapy to administer to the subject based on the identification of the dominant copy number signature or of the one or more copy number signature components. In some embodiments, the method further comprises administering the anti-cancer therapy to the subject based on the identification of the dominant copy number signature or of the one or more copy number signature components. In some embodiments, the anti-cancer therapy comprises chemotherapy, radiation therapy, immunotherapy, a targeted therapy, or surgery.

Disclosed herein are methods for diagnosing a disease, the method comprising: diagnosing that a subject has the disease based on an identification of a dominant copy number signature or of one or more copy number signature components in a copy number profile for a sample from an individual, wherein the dominant copy number signature or the one or more copy number signature components are identified according to any of the methods described herein.

Disclosed herein are methods of selecting an anti-cancer therapy, the method comprising: responsive to an identification of a dominant copy number signature or of one or more copy number signature components in a copy number profile for a sample from an individual, selecting an anti-cancer therapy for the individual, wherein the dominant copy number signature or the one or more copy number signature components are identified according to any of the methods described herein.

Disclosed herein are methods of treating a cancer in an individual, comprising: responsive to an identification of a dominant copy number signature or of one or more copy number signature components in a copy number profile for a sample from an individual, administering an effective amount of an anti-cancer therapy to the individual, wherein the dominant copy number signature or the one or more copy number signature components are identified according to any of the methods described herein.

Disclosed herein are methods for monitoring cancer progression or recurrence in an individual, the method comprising: identifying a first dominant copy number signature, or a first set of one or more copy number signature components, in a first copy number profile for a first sample obtained from an individual at a first time point according to any of the methods described herein; identifying a second dominant copy number signature, or a second set of one or more copy number signature components, in a second copy number profile for a second sample obtained from the individual at a second time point; and comparing the first dominant copy number signature, or the first set of one or more copy number signature components, to the second dominant copy number signature, or the second set of one or more copy number signature components, thereby monitoring the cancer progression or recurrence. In some embodiments, the second dominant copy number signature, or the second set of one or more copy number signature components, for the second sample is determined according to any of the methods described herein. In some embodiments, the method further comprises selecting an anti-cancer therapy for the individual in response to the cancer progression. In some embodiments, the method further comprises administering an anti-cancer therapy to the individual in response to the cancer progression. In some embodiments, the method further comprises adjusting an anti-cancer therapy for the individual in response to the cancer progression. In some embodiments, the method further comprises adjusting a dosage of the anti-cancer therapy or selecting a different anti-cancer therapy in response to the cancer progression. In some embodiments, the method further comprises administering the adjusted anti-cancer therapy to the individual. In some embodiments, the first time point is before the individual has been administered an anti-cancer therapy, and wherein the second time point is after the individual has been administered the anti-cancer therapy. In some embodiments, the individual has a cancer, is at risk of having a cancer, is being routine tested for cancer, or is suspected of having a cancer. In some embodiments, the cancer is a solid tumor. In some embodiments, the cancer is a hematological cancer. In some embodiments, the anti-cancer therapy comprises chemotherapy, radiation therapy, immunotherapy, a targeted therapy, or surgery.

In some embodiments of any of the methods described herein, the method further comprises determining, identifying, or applying the dominant copy number signature, or the one or more copy number signature components, for the sample as a diagnostic value associated with the sample. In some embodiments, the method further comprises generating a genomic profile for the individual based on the identification of a dominant copy number signature or of one or more copy number signature components in a copy number profile for the sample from the individual. In some embodiments, the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the individual further comprises results from a nucleic acid sequencing-based test. In some embodiments, the method further comprises selecting an anti-cancer therapy, administering an anti-cancer therapy, or applying an anti-cancer therapy to the individual based on the generated genomic profile. In some embodiments, the identification of a dominant copy number signature or of one or more copy number signature components in a copy number profile for the sample from the individual is used in making suggested treatment decisions for the subject. In some embodiments, the identification of a dominant copy number signature or of one or more copy number signature components in a copy number profile for the sample from the individual is used in applying or administering a treatment to the subject.

Disclosed herein are methods for identifying an individual that may benefit from treatment with a poly (ADP-ribose) polymerase inhibitor (PARPi), comprising: receiving, at one or more processors, targeted sequence read data for a sample derived from an individual diagnosed with or suspected of having cancer; determining, using the one or more processors, a copy number profile having a plurality of copy number features for the individual based on the targeted sequence read data for the sample; determining, using the one or more processors, a copy number feature value for each of the plurality of copy number features; performing, using the one or more processors, an analysis of the copy number feature values for each of a plurality of copy number features using a first trained model to determine a probability that each of one or more copy number feature subgroups is represented in the copy number profile for the sample; performing, using the one or more processors, an analysis of the probabilities determined for each of the one or more copy number feature subgroups using a second trained model to identify one or more copy number signatures represented in the copy number profile for the individual, and to determine one or more corresponding copy number signature scores; comparing, using the one or more processors, the copy number signature scores for the one or more identified copy number signatures to one or more corresponding predetermined thresholds; and

identifying, based on the comparison, a dominant copy number signature for the individual, wherein the dominant copy number signature comprises a homologous recombination deficient (HRD) signature; or identifying, based on the comparison, one or more copy number signatures that are components of the copy number profile for the individual, wherein the one or more copy number signatures comprise a homologous recombination deficient (HRD) signature; wherein identification of the HRD signature indicates that the individual may benefit from treatment with a PARPi.

In some embodiments, the method further comprises administering an effective amount of a PARPi to the individual. In some embodiments, the PARPi comprises olaparib, rucaparib, niraparib, talazoparib, or veliparib. In some embodiments, the cancer is ovarian cancer. In some embodiments, the cancer is prostate cancer.

In some embodiments, the method further comprising, prior to receiving the targeted sequence read data: providing a plurality of nucleic acid molecules obtained from a sample from the individual; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; and sequencing, by a sequencer, the captured nucleic acid molecules to obtain a plurality of sequence reads that represent the captured nucleic acid molecules.

In some embodiments, the sample lacks a loss-of-function mutation in BRCA1 and/or BRCA2 gene(s). In some embodiments, the sample comprises biallelic loss-of-function mutations in one or more of BARD1, PALB2, RAD51D, and RAD51C gene(s). In some embodiments, the sample further lacks a loss-of-function mutation in ATM, BARD1, BRIP1, CDK12, CHEK1, CHEK2, FANCL, PALB2, RAD51B, RAD51C, RAD51D, and RAD54L gene(s).

In some embodiments, the HRD signature comprises a plurality of low copy number segments. In some embodiments, the HRD signature comprises heterozygous deletion(s), high genome-wide loss of heterozygosity (gLOH-high), and/or high genomic instability score (GIS).

In some embodiments, the cancer is ovarian or breast cancer, and the HRD signature comprises heterozygous deletion(s), heterozygous duplication(s), and high genome-wide loss of heterozygosity (gLOH-high). In some embodiments, the cancer is prostate cancer, and the HRD signature comprises heterozygous deletion(s) and copy neutral loss of heterozygosity.

In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

Also disclosed herein are systems comprising: one or more processors; and a memory communicatively coupled to the one or more processors and configured to store instructions that, when executed by the one or more processors, cause the system to: receive targeted sequence read data for a sample derived from an individual diagnosed with or suspected of having a disease; determine a copy number profile having a plurality of copy number features for the individual based on the targeted sequence read data for the sample; determine a copy number feature value for each of the plurality of copy number features; perform an analysis of the copy number feature values for each of a plurality of copy number features using a first trained model to determine a probability that each of one or more copy number feature subgroups is represented in the copy number profile for the sample; perform an analysis of the probabilities determined for each of the one or more copy number feature subgroups using a second trained model to identify one or more copy number signatures represented in the copy number profile for the individual, and to determine one or more corresponding copy number signature scores; compare the copy number signature scores for the one or more identified copy number signatures to one or more corresponding predetermined thresholds; and identify, based on the comparison, a dominant copy number signature for the individual; or identify, based on the comparison, one or more copy number signatures that are components of the copy number profile for the individual.

In some embodiments, the copy number profile for the individual is determined based on off-target sequence reads in the targeted sequence read data for the sample. In some embodiments, the copy number profile for the individual is determined based on single nucleotide polymorphisms (SNPs) located in the targeted sequence read data for the sample. In some embodiments, the single nucleotide polymorphisms (SNPs) are located in on-target sequence read data. In some embodiments, the copy number profile for the individual is determined based on off-target sequence reads in the targeted sequence read data for the sample.

In some embodiments, the copy number profile for the individual is determined based on a combination of on-target and off-target sequence reads in the targeted sequence read data for the sample. In some embodiments, the off-target sequence reads comprise an average coverage of less than 0.5×, 0.4×, 0.3×, 0.2×, or 0.1×.

In some embodiments, the analysis to identify one or more copy number signatures represented in the copy number profile of the individual comprises an analysis of both the probabilities determined for each of the one or more copy number feature subgroups and a presence of one or more genomic features detected in the targeted sequence read data for the individual using the second trained model. In some embodiments, the one or more genomic features comprise one or more short variants, one or more indels, one or more biallelic loss-of-function variants, one or more rearrangements, or any combination thereof.

In some embodiments, the plurality of copy number features comprises a number of breakpoints per 10 Mb of genomic sequence, a number of breakpoints per 25 Mb of genomic sequence, a number of breakpoints per 50 Mb of genomic sequence, a number of breakpoints per 100 Mb of genomic sequence, a number of breakpoints per chromosome arm for each chromosome, a magnitude of a copy number change between any two adjacent copy number segments; a length of each copy number segment; the copy number of segments; a count of contiguous oscillating copy number chains, or any combination thereof.

In some embodiments, the first trained model comprises a trained statistical model. In some embodiments, the first trained model comprises a trained machine learning model. In some embodiments, the first trained model is updated based on the one or more copy number signatures identified in the copy number profile of the individual. In some embodiments, the first trained model comprises a latent class analysis model or a mixture model.

In some embodiments, the second trained model comprises a non-negative matrix factorization (NMF) model or a machine learning model. In some embodiments, the second trained model comprises a machine learning model, and the machine learning model comprises a supervised, semi-supervised, or unsupervised machine learning model. In some embodiments, the second trained model comprises a supervised machine learning model, and the supervised machine learning model comprises an XGBoost model.

In some embodiments, the one or more corresponding predetermined thresholds are determined based on an analysis of copy number signature scores for a cohort of individuals diagnosed with the disease. In some embodiments, the one or more corresponding predetermined thresholds are determined based on an analysis of copy number signature scores for a cohort of individuals comprising individuals diagnosed with a plurality of different diseases. In some embodiments, the one or more corresponding predetermined thresholds are determined based on an analysis of data for a cohort of individuals that comprises both copy number signature scores and clinical data.

Disclosed herein are non-transitory computer-readable storage media storing one or more programs, the one or more programs comprising instructions, which when executed by one or more processors of a system, cause the system to: receive targeted sequence read data for a sample derived from an individual diagnosed with or suspected of having a disease; determine a copy number profile having a plurality of copy number features for the individual based on the targeted sequence read data for the sample; determine a copy number feature value for each of a plurality of copy number features; perform an analysis of the copy number feature values for each of a plurality of copy number features using a first trained model to determine a probability that each of one or more copy number feature subgroups is represented in the copy number profile for the sample; perform an analysis of the probabilities determined for each of the one or more copy number feature subgroups using a second trained model to identify one or more copy number signatures represented in the copy number profile for the individual, and to determine one or more corresponding copy number signature scores; compare the copy number signature scores for the one or more identified copy number signatures to one or more corresponding predetermined thresholds; and identify, based on the comparison, a dominant copy number signature for the individual; or identify, based on the comparison, one or more copy number signatures that are components of the copy number profile for the individual.

In some embodiments, the copy number profile for the individual is determined based on off-target sequence reads in the targeted sequence read data for the sample. In some embodiments, the copy number profile for the individual is determined based on single nucleotide polymorphisms (SNPs) located in the targeted sequence read data for the sample. In some embodiments, the single nucleotide polymorphisms (SNPs) are located in on-target sequence read data. In some embodiments, the copy number profile for the individual is determined based on off-target sequence reads in the targeted sequence read data for the sample.

In some embodiments, the copy number profile for the individual is determined based on a combination of on-target and off-target sequence reads in the targeted sequence read data for the sample. In some embodiments, the off-target sequence reads comprise an average coverage of less than 0.5×, 0.4×, 0.3×, 0.2×, or 0.1×.

In some embodiments, the analysis to identify one or more copy number signatures represented in the copy number profile of the individual comprises an analysis of both the probabilities determined for each of the one or more copy number feature subgroups and a presence of one or more genomic features detected in the targeted sequence read data for the individual using the second trained model. In some embodiments, the one or more genomic features comprise one or more short variants, one or more indels, one or more biallelic loss-of-function variants, one or more rearrangements, or any combination thereof.

In some embodiments, the plurality of copy number features comprises a number of breakpoints per 10 Mb of genomic sequence, a number of breakpoints per 25 Mb of genomic sequence, a number of breakpoints per 50 Mb of genomic sequence, a number of breakpoints per 100 Mb of genomic sequence, a number of breakpoints per chromosome arm for each chromosome, a magnitude of a copy number change between any two adjacent copy number segments; a length of each copy number segment; the copy number of segments; a count of contiguous oscillating copy number chains, or any combination thereof.

In some embodiments, the first trained model comprises a trained statistical model. In some embodiments, the first trained model comprises a trained machine learning model. In some embodiments, the first trained model is updated based on the one or more copy number signatures identified in the copy number profile of the individual. In some embodiments, the first trained model comprises a latent class analysis model or a mixture model.

In some embodiments, the second trained model comprises a non-negative matrix factorization (NMF) model or a machine learning model. In some embodiments, the second trained model comprises a machine learning model, and the machine learning model comprises a supervised, semi-supervised, or unsupervised machine learning model. In some embodiments, the second trained model comprises a supervised machine learning model, and the supervised machine learning model comprises an XGBoost model.

In some embodiments, the one or more corresponding predetermined thresholds are determined based on an analysis of copy number signature scores for a cohort of individuals diagnosed with the disease. In some embodiments, the one or more corresponding predetermined thresholds are determined based on an analysis of copy number signature scores for a cohort of individuals comprising individuals diagnosed with a plurality of different diseases. In some embodiments, the one or more corresponding predetermined thresholds are determined based on an analysis of data for a cohort of individuals that comprises both copy number signature scores and clinical data.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for identifying one or more copy number signatures present in a copy number profile determined from targeted sequencing data for a sample collected from an individual (e.g., a cancer patient), according to one embodiment described herein.

FIG. 2 provides a non-limiting example of a process flowchart for training a first model and a second model for use in identifying one or more copy number signatures present in a copy number profile determined from targeted sequencing data for a sample collected from an individual (e.g., a cancer patient), according to one embodiment described herein.

FIG. 3 depicts an exemplary computing device or system in accordance with one embodiment of the present disclosure.

FIG. 4 depicts an exemplary computer system or computer network, in accordance with some instances of the systems described herein.

FIGS. 5A-5F show that HRDsig predicts PARPi benefit in ovarian and prostate clinical cohorts. FIG. 5A: Ovary CGDB cohort selection schematic. FIG. 5B: Kaplan-Meier curves showing HRDsig as predictor of response to PARPi treatment in ovarian cancer. FIG. 5C: Multivariate analysis of predictors of response in ovarian cancer. FIG. 5D: Prostate CGDB cohort selection schematic. FIG. 5E: Kaplan-Meier curves showing HRDsig as predictor of response to PARPi treatment in prostate cancer. FIG. 5F: Multivariate analysis of predictors of response in prostate cancer.

FIGS. 6A-6F show the landscape of copy number signatures in a pan-cancer cohort. FIG. 6A: Stacked bar plot showing the prevalence of copy number signatures across 46 tumor types. Low aneuploidy signature was excluded from this plot due to it being characterized as a low aberrant copy number profile. FIG. 6B: Bar plot depicting the prevalence of the low aneuploidy signature across tumor types. FIG. 6C: The size of each dot represents the prevalence of each signature in each tumor type. The color of dot represents the enrichment (red) or depletion (blue) of the signatures in each tumor type. Enrichment or depletion was determined using Fisher's exact test and the odd's ratio (OR) was log 2 normalized. FIG. 6D: The size of each dot represents the median sample segment copy number across signatures and tumor types. The color of the dot represents the −log 10 transformed FDR-corrected P value. FIG. 6E: A plot showing the enrichment of biomarker positive calls in signature positive samples. The size of each dot represents the prevalence of each biomarker positive call in each signature positive cohort. The color of dot represents the enrichment (red) or depletion (blue) of the biomarker positive call in each copy number signature positive cohort. Enrichment or depletion was determined using Fisher's exact test and the odd's ratio (OR) was log 2 normalized. FIG. 6F: A plot showing the enrichment of short variant mutational signatures in copy number signature positive samples. The size of each dot represents the prevalence of mutational signature positive calls in copy number signature positive cohorts. The y-axis represents the −log 10 transformed FDR-corrected P value. Mutational signatures above the horizontal dotted line are significant (FDR P value <0.05). The x-axis represents the log 2 normalized OR value, where dots to the right of the central log 2 OR=0 line are enriched (OR>1), and dots to the left of that line are depleted (OR<1).

FIG. 7 shows descriptions of 10 copy number signatures identified in pan-cancer samples.

FIG. 8 shows an exemplary copy number profile for each copy number signature. Each sample's tumor type and relevant genomics are included.

FIG. 9 shows the percent change in mean for features in samples with a signature compared to samples without the signature. Mann-Whitney U test result −log 10 transformed Pval of percent change in continuous genomic features compared in samples with and without the signature.

FIG. 10 shows that high amounts of FTD events occur across diseases with chromosomal instability signatures. The size of each dot represents the prevalence of each copy number signature called positive in each disease group. The color of the dot represents the mean number of FTD events overserved in samples in each disease group and copy number signature positive group.

FIGS. 11A & 11B show genomic associations with copy number signatures. FIG. 11A: A plot showing the enrichment of gene alterations in copy number signatures. The size of each dot represents the prevalence of the genes being altered in copy number signature positive samples. The y-axis represents the −log 10 transformed FDR-corrected P value. Dots above the horizontal dotted line are significant (FDR P value <0.05). The x-axis represents the log 2 normalized OR value, where dots to the right of the central log 2 OR=0 line are enriched (OR>1), and dots to the left of that line are depleted (OR<1). FIG. 11B: A plot showing the enrichment of cytogenetic events (arm level gain or losses) across signatures. The size of each dot represents the prevalence of the cytogenetic arm level event occurring in copy number signature positive samples. The y-axis represents the −log 10 transformed FDR-corrected P value. Dots above the horizontal dotted line are significant (FDR P value <0.05). The x-axis represents the log 2 normalized OR value, where dots to the right of the central log 2 OR=0 line are enriched (OR>1), and dots to the left of that line are depleted (OR<1).

FIGS. 12A-12F show that HRD copy number signatures capture genomic scarring (e.g., BRCAness). FIG. 12A: A schematic of model training and evaluation. FIG. 12B: Performance of HRDsig and gLOH models in predicting approximate true positive (biallelic BRCA1/2) and approximate true negative (HRRwt) in all tumor types. FIG. 12C: HRDsig probability plotted for each disease, broken down by biallelic BRCA samples (red), other mutated HRR (other HRRm) (grey), and HRRwt (yellow). FIG. 12D: Frequency of HRD positive calls in disease groups. Stacked bars of percentage of biallelic BRCA, other HRRm or HRRwt prevalence within positively called group. FIG. 12E: HRDsig sensitivity in detecting biallelic BRCA1/2 alerations in all samples, as well as in key diseases. FIG. 12F: Gene alteration enrichment in BRCAwt samples based on HRDsig status. Y-axis is limited to −log 10 P-value <80.

FIG. 13 shows that HRDsig is called at a similar rate in liquid biopsy as compared to tissue biopsy. Scatterplot shows the prevalence of HRDsig-positive calls in tissue biopsy and prevalence of HRDsig-positive calls in liquid biopsy samples. A cutoff of >50 assessable liquid biopsy samples was used. Size of the points is the number of assessable liquid biopsies in each tumor type.

FIG. 14 shows a comparison of PARPi treated ovarian cancer patients at treatment start.

FIG. 15 shows HRD stratification in PARPi response in BRCA1/2 wildtype ovarian cancer (CGDB).

FIG. 16 shows a comparison of PARPi treated prostate cancer patients at treatment start.

FIG. 17 shows time-to-treatment discontinuation (TTD) in ovarian cancer for genome-wide loss of heterozygosity (gLOH) and genomic instability score (GIS). Kaplan-Meier curves show that gLOH (≥16%) and GIS (≥42) were associated with significant PARPi TTD (gLOH HR 0.49 [0.33-0.72]p<0.001; GIS HR 0.69 [0.50-0.97]p=0.03) in ovarian cancer.

FIG. 18 shows TTD in prostate cancer for gLOH and GIS. Kaplan-Meier curves show that gLOH and GIS were associated with PARPi TTD (gLOH-high: HR 0.53 [0.24-1.18]p=0.3; GIS>42: HR 0.44 [0.17-1.09]p=0.08) in prostate cancers.

### DETAILED DESCRIPTION

Methods and systems are described for identifying copy number signature(s) in a copy number profile for a sample from an individual (e.g., a patient), and for identifying the dominant copy number signature, or a set of one or more copy number signatures that constitute significant components of the copy number profile for the individual. Advantages of the disclosed methods and systems over previous work include utilization of off-target sequence read data derived from a targeted sequencing assay to maximize coverage of copy number features while minimizing sequencing costs, and the identification of an expanded set of copy number signatures as compared to those described in previous work.

In some instances, for example, methods are described that comprise receiving targeted sequence read data for a sample derived from an individual diagnosed with or suspected of having a disease; determining a copy number profile having a plurality of copy number features for the individual based on the targeted sequence read data for the sample; determining a copy number feature value for each of the plurality of copy number features; performing an analysis of the copy number feature values for each of a plurality of copy number features using a first trained model to determine a probability that each of one or more copy number feature subgroups is represented in the copy number profile for the sample; performing an analysis of the probabilities determined for each of the one or more copy number feature subgroups using a second trained model to identify one or more copy number signatures represented in the copy number profile for the individual, and to determine one or more corresponding copy number signature scores; comparing the copy number signature scores for the one or more identified copy number signatures to one or more corresponding predetermined thresholds; and identifying, based on the comparison, a dominant copy number signature for the individual; or identifying, based on the comparison, one or more copy number signatures that are components of the copy number profile for the individual.

In some instances, the method further comprises confirming a diagnosis of the disease for the individual based on the dominant copy number signature or the one or more copy number signatures identified for the individual. In some instances, the method further comprises predicting a treatment outcome for the individual based on the dominant copy number signature or the one or more copy number signatures identified for the individual. In some instances, the method further comprises selecting a treatment for the individual based on the dominant copy number signature or the one or more copy number signatures identified for the individual. In some instances, the method further comprises adjusting a treatment dosage for the individual based on the dominant copy number signature or the one or more copy number signatures identified for the individual. In some instances, the method further comprises selecting the individual for inclusion in a clinical trial based on the dominant copy number signature or the one or more copy number signatures identified for the individual. In some instances, the method further comprises identifying the individual as a candidate for chemotherapy, radiation therapy, or surgery based on the dominant copy number signature or the one or more copy number signatures identified for the individual. In some instances, the method further comprises identifying the individual as a candidate for hospice care based on the dominant copy number signature or the one or more copy number signatures identified for the individual.

In some instances, the copy number profile for the individual is determined based on single nucleotide polymorphisms (SNPs) located in the targeted sequence read data for the sample. In some instances, the single nucleotide polymorphisms (SNPs) are located in on-target sequence read data. In some instances, the copy number profile for the individual is determined based on off-target sequence reads in the targeted sequence read data for the sample. In some instances, the copy number profile for the individual is determined based on a combination of on-target and off-target sequence reads in the targeted sequence read data for the sample.

In some instances, the plurality of copy number features comprises a number of breakpoints per 10 Mb of genomic sequence, a number of breakpoints per 25 Mb of genomic sequence, a number of breakpoints per 50 Mb of genomic sequence, a number of breakpoints per 100 Mb of genomic sequence, a number of breakpoints per chromosome arm for each chromosome, a magnitude of a copy number change between any two adjacent copy number segments; a length of each copy number segment; the copy number of segments; a count of contiguous oscillating copy number chains, or any combination thereof.

In some instances, the first trained model comprises a trained statistical model. In some instances, the first trained model comprises a trained machine learning model. In some instances, the first trained model is updated based on the one or more copy number signatures identified in the copy number profile of the individual. In some instances, the first trained model comprises a latent class analysis model or a mixture model.

In some instances, the second trained model comprises a non-negative matrix factorization (NMF) model or a machine learning model. In some instances, the second trained model comprises a machine learning model, and the machine learning model comprises a supervised, semi-supervised, or unsupervised machine learning model. In some instances, the second trained model comprises a supervised machine learning model, and the supervised machine learning model comprises an XGBoost model.

As noted above, advantages of the disclosed methods and systems over previous work include utilization of off-target sequence read data derived from a targeted sequencing assay to maximize coverage of copy number features while minimizing sequencing costs, and the identification of an expanded set of copy number signatures as compared to those described in previous work.

### DEFINITIONS

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

Throughout this application, various parameter values may be presented in a range format. It should be understood that the description in range format is merely for convenience and brevity, and should not be construed as an inflexible limitation on the scope of the disclosure. Accordingly, the description of a range should be considered to have specifically disclosed all possible subranges as well as individual numerical values within that range, irrespective of whether a specific numerical value or specific sub-range is expressly stated. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 1.4, 2, 3, 3.6, 4, 5, 5.8, and 6. This applies regardless of the breadth of the range.

Numbers may be expressed herein as being “about” a particular value. Similarly, ranges may be expressed herein as from “about” one particular value and/or to “about” another particular value. The terms “about” and “approximately” shall generally mean an acceptable degree of error or variation for a given value or range of values, such as, for example, a degree of error or variation that is within 20 percent (%), within 15%, within 10%, or within 5% of a given value or range of values.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “on-target sequence read” refers to a sequence read corresponding to a genomic locus targeted by a bait molecule or other locus-specific enrichment technique used in a targeted sequencing assay.

As used herein, the term “off-target sequence read” refers to a sequence read corresponding to a genomic locus that is not targeted by a bait molecule or other locus-specific enrichment technique used in a targeted sequencing assay. Off-target sequence reads may arise, for example, due to the inefficiencies of enrichment strategies used for targeted sequencing.

As used herein, the term “breakpoint” refers to a genomic region or locus where a sample genomic sequence may have a different copy number level than an adjacent segment. These may include sites of breakage where a chromosome breaks (and recombines).

As used herein, the term “breakpoint count per section of genomic sequence” represents the number of breakpoints per a specified section of genomic sequence across the genome or a portion of the genome. For example, to determine the breakpoint count per 10 megabases (Mb) of sequence, a series of adjacent processing windows (or alternatively, a sliding processing window) of 10 Mb is analyzed for the genome, or a portion thereof, and the number of breakpoints for each frame of the processing window can then be assessed.

As used herein, the term “change point copy number” refers to the absolute difference in copy number between two adjacent genome segments. For example, adjacent segments modeled at copy numbers of 7 and 2 would have an absolute different—or change point copy number—of 5.

As used herein, the term “copy number profile” refers to a vector u=(u₁, u₂, . . . , uₙ) of non-negative integers representing the copy-number of each position (or contiguous segment) in a genome, where changes in copy number are generated by, e.g., amplification and deletion events.

As used herein, the term “copy number signature” refers to a pattern of copy number alterations accumulated across a genome that, in turn, are based on patterns of copy number features present in the genome.

As used herein, the term “copy number oscillation” refers to copy number patterns in the DNA, such as repeating copy number patterns, that may arise through various processes including, but not limited to, chromothripsis. The number of segments with oscillating copy number represents a traversal of the genome, or a portion thereof, while counting the number of repeated alternating segments between two copy numbers.

As used herein, the term “chromothripsis” refers to a mutational process in which a large number of clustered chromosomal rearrangements occur in a single event in localized and confined genomic regions in one or a few chromosomes. Chromothripsis is known to be involved in both cancer and congenital diseases.

It should be recognized that use of ordinal terms such as “first” and “second” in the description of methods and systems disclosed herein does not by itself connote any priority, order of importance of one system component over another, or the temporal order in which acts of a method are performed, but are used merely as labels to distinguish, for example, one system component having a certain name from another system component having the same name but for the use of the ordinal term to distinguish the two system components.

Additionally, various implementations of the methods and systems set forth herein may be described in terms of exemplary block diagrams, process flow charts, and other illustrations. As will become apparent to one of ordinary skill in the art after reading this document, the various implementations set forth herein can be implemented without confinement to the illustrated examples. For example, block diagrams and their accompanying description should not be construed as mandating a particular architecture or configuration. Similarly, in exemplary process flow charts, some blocks are optionally combined, the order of some blocks is optionally changed, and some blocks are optionally omitted. In some implementations, additional steps may be performed in combination with the exemplary processes. Accordingly, the methods and systems as described and illustrated in greater detail below are exemplary by nature and, as such, should not be viewed as limiting.

The section headings used herein are for organizational purposes only and are not to be construed as limiting the subject matter described.

### Methods for Determining Copy Number Signatures

The objective of the methods described herein is to determine a copy number profile and evaluate copy number features identified in genomic DNA extracted from a sample (e.g., a biopsy sample) from an individual (e.g. a patient) based on targeted sequencing data, and to use those evaluated copy number features to identify copy number signatures present in the individual's genomic copy number profile that may represent important genomic characteristics, such as the presence of seismic amplifications (characterized by multiple rearrangements and discontinuous copy number levels), chromothripsis (characterized by a large number of clustered chromosomal rearrangements that occur in a single event in localized and confined genomic regions in one or a few chromosomes), etc.

In some instances, the disclosed methods utilize off-target coverage of genomic loci (e.g., non-targeted single nucleotide polymorphisms (SNPs)) from targeted sequencing data to derive copy number features for a genomic sample. Off-target coverage relies on the presence of off-target sequence reads in the targeted sequence read data due to, for example, the inefficiencies of the enrichment strategies used for performing the targeted sequencing. In some instances, the disclosed methods may utilize a combination of off-target and on-target coverage of genomic loci in targeted sequencing data. In some instances, the disclosed methods may utilize other sequencing data sets, e.g., whole genome sequencing data, low pass whole genome sequencing data, whole exome sequencing data, etc.

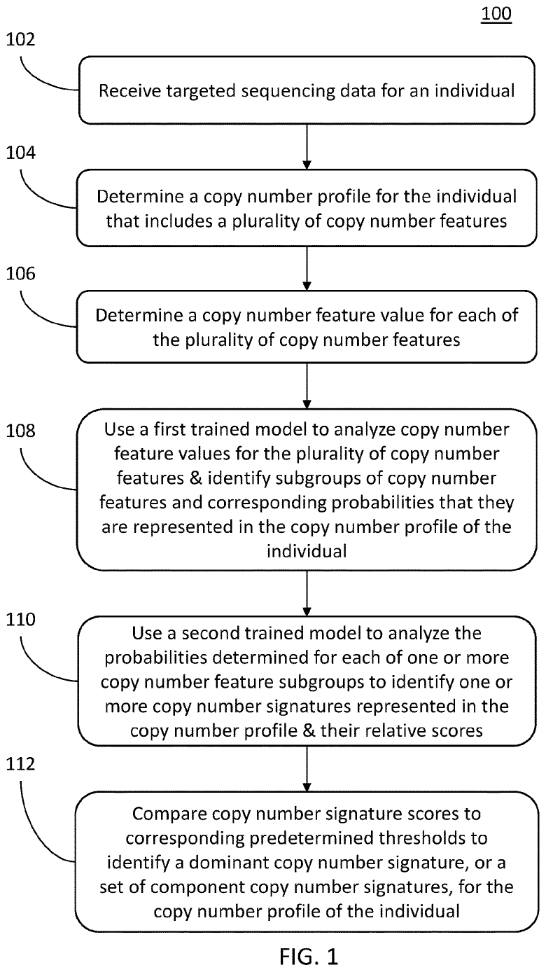

FIG. 1 provides a non-limiting example of a flowchart for a process 100 for identifying one or more copy number signatures present in a copy number profile determined from targeted sequencing data for a sample collected from an individual (e.g., a cancer patient). Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, a set of targeted sequencing data—derived by sequencing nucleic acid molecules (e.g., DNA) extracted from a sample collected from an individual (e.g., a patient)—is received. Methods for collecting samples from individual, extracting nucleic acid molecules therefrom, and sequencing nucleic acid molecules are described elsewhere herein.

In some instances, the sample may be a solid biopsy sample (e.g., a tumor sample). In some instances, the sample may be a liquid biopsy sample, and may comprise, e.g., whole blood, blood plasma, blood serum, urine, stool, sputum, saliva, or cerebrospinal fluid. In some instances, the sample may be a liquid biopsy sample and may comprise cell-free DNA (cfDNA), circulating tumor DNA (ctDNA), or any combination thereof.

In some instances, the generation of targeted sequencing data may comprise use of a next-generation sequencing as described elsewhere herein. As noted above, in some instances, the disclosed methods may be applied to non-targeted sequencing data, e.g., whole genome sequencing data, low pass whole genome sequencing data, whole exome sequencing data, etc.

At step 104 in FIG. 1, a copy number profile is determined for the individual, where the copy number profile can include a plurality of copy number features. Copy number profile determination may be performed using any of a variety of segmentation and/or copy number modeling techniques known to those of skill in the art, e.g., sliding window techniques and hidden Markov Model (HMM) techniques (see, e.g., Wang, et al. (2014), “Copy Number Variation Detection Using Next Generation Sequencing Read Counts”, BMC Bioinformatics 15:109).

In some instances of the disclosed methods, the copy number profile for the individual may be determined based on an analysis of off-target sequence reads in the targeted sequence read data for the sample. In some instances, the copy number profile for the individual may be determined based on an analysis of on-target sequence reads in the targeted sequence read data for the sample. In some instances, the copy number profile for the individual may be determined based on a combination of on-target and off-target sequence reads in the targeted sequence read data for the sample.

In some instances of the disclosed methods, the copy number profile for the individual is determined based on single nucleotide polymorphisms (SNPs) located in the targeted sequence read data for the sample. In some instances, the SNPs are located in the off-target sequence reads of the targeted sequence read data set. In some instances, the SNPs are located in the on-target sequence reads of the targeted sequence read data set. In some instances, the SNPs are located in a combination of off-target and on-target sequence reads of the targeted sequence read data set.

As noted above, off-target sequence reads may arise in a targeted sequence read data set due to, for example, the inefficiencies of the enrichment strategies used for performing the targeted sequencing. In some instances, the off-target sequence reads may comprise an average sequencing coverage of less than 0.5×, 0.4×, 0.3×, 0.2×, or 0.1×.

In some instances, the copy number profile determined for an individual may comprise one or more copy number features (i.e., key attributes of the copy number profile). Examples of copy number features are described in more detail below. In some instances, the copy number profile may comprise at least 1, at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, or more than 80 copy number features.

At step 106 in FIG. 1, a copy number value for each of the plurality of copy number features is determined. Examples of copy number features to be evaluated include, but are not limited to, the number of breakpoints per 10 Mb of genomic sequence, the number of breakpoints per 25 Mb of genomic sequence, the number of breakpoints per 50 Mb of genomic sequence, the number of breakpoints per 100 Mb of genomic sequence, the number of breakpoints per chromosome arm for each chromosome, the magnitude of a copy number change between any two adjacent copy number segments; the length of each copy number segment; the copy number of each segment; change point copy number, the number of contiguous oscillating copy number chains, the length of oscillating copy number chains, segment minority allele frequency, or any combination thereof.

In some instances, the copy number features may comprise features that address whole genome data, centromeric data, telomeric data, or any combination thereof. In some instances, the copy number features to be evaluated may include, but are not limited to, bp10MB1, bp10MB2, bp10MB3, bp10MB4, bp10MB5, bp10MB6, bp100MB1, bp100MB2, bp100MB3, bp100MB4, bp100MB5, bp100MB6, bp50MB1, bp50MB2, bp50MB3, bp50MB4, bp50MB5, bp50MB6, bp25MB1, bp25MB2, bp25MB3, bp25MB4, bp25MB5, bp25MB6, segsize1, segsize2, segsize3, segsize4, segsize5, segsize6, segsize7, segsize8, segsize9, segsize10, segsize11, segsize12, segsize13, osCN1, osCN2, osCN3, osCN4, osCN5, osCN6, bpchrarm1, bpchrarm2, bpchrarm3, copynumber1, copynumber2, copynumber3, copynumber4, copynumber5, copynumber6, copynumber7, copynumber8, copynumber9, copynumber10, copynumber11, copynumber12, copynumber13, copynumber14, copynumber15, changepoint1, changepoint2, changepoint3, changepoint4, changepoint5, changepoint6, changepoint7, changepoint8, changepoint9, changepoint10, changepoint11, changepoint12, changepoint13, changepoint14, changepoint15, bp10MB1_centromeric, bp10MB2_centromeric, bp10MB3_centromeric, bp10MB4_centromeric, bp10MB5_centromeric, bp10MB6_centromeric, bp100MB1_centromeric, bp100MB2_centromeric, bp100MB3_centromeric, bp100MB4_centromeric, bp100MB5_centromeric, bp100MB6_centromeric, bp50MB1_centromeric, bp50MB2_centromeric, bp50MB3_centromeric, bp50MB4_centromeric, bp50MB5_centromeric, bp50MB6_centromeric, bp25MB1_centromeric, bp25MB2_centromeric, bp25MB3_centromeric, bp25MB4_centromeric, bp25MB5_centromeric, bp25MB6_centromeric, segsize1_centromeric, segsize2_centromeric, segsize3_centromeric, segsize4_centromeric, segsize5_centromeric, segsize6_centromeric, segsize7_centromeric, segsize8_centromeric, segsize9_centromeric, segsize10_centromeric, segsize11_centromeric, segsize12_centromeric, segsize13_centromeric, segsize14_centromeric, osCN1_centromeric, osCN2_centromeric, bpchrarm1_centromeric, bpchrarm2_centromeric, bpchrarm3_centromeric, bpchrarm4_centromeric, bpchrarm5_centromeric, bpchrarm6_centromeric, copynumber1_centromeric, copynumber2_centromeric, copynumber3_centromeric, copynumber4_centromeric, copynumber5_centromeric, copynumber6_centromeric, copynumber7_centromeric, copynumber8_centromeric, copynumber9_centromeric, copynumber10_centromeric, copynumber11_centromeric, copynumber12_centromeric, copynumber13_centromeric, copynumber14_centromeric, copynumber15_centromeric, changepoint1_centromeric, changepoint2_centromeric, changepoint3_centromeric, changepoint4_centromeric, changepoint5_centromeric, changepoint6_centromeric, changepoint7_centromeric, changepoint8_centromeric, changepoint9_centromeric, changepoint10_centromeric, changepoint11_centromeric, changepoint12_centromeric, changepoint13_centromeric, changepoint14_centromeric, changepoint15_centromeric, bp10MB1_telomeric, bp10MB2_telomeric, bp10MB3_telomeric, bp10MB4_telomeric, bp10MB5_telomeric, bp10MB6_telomeric, bp100MB1_telomeric, bp100MB2_telomeric, bp100MB3_telomeric, bp100MB4_telomeric, bp100MB5_telomeric, bp100MB6_telomeric, bp50MB1_telomeric, bp50MB2_telomeric, bp50MB3_telomeric, bp50MB4_telomeric, bp50MB5_telomeric, bp50MB6_telomeric, bp25MB1_telomeric, bp25MB2_telomeric, bp25MB3_telomeric, bp25MB4_telomeric, bp25MB5_telomeric, bp25MB6_telomeric, segsize1_telomeric, segsize2_telomeric, segsize3_telomeric, segsize4_telomeric, segsize5_telomeric, segsize6_telomeric, segsize7_telomeric, segsize8_telomeric, segsize9_telomeric, segsize10_telomeric, segsize11_telomeric, segsize12_telomeric, segsize13_telomeric, segsize14_telomeric, segsize15_telomeric, osCN1_telomeric, osCN2_telomeric, bpchrarm1_telomeric, bpchrarm2_telomeric, bpchrarm3_telomeric, bpchrarm4_telomeric, bpchrarm5_telomeric, bpchrarm6_telomeric, copynumber1_telomeric, copynumber2_telomeric, copynumber3_telomeric, copynumber4_telomeric, copynumber5_telomeric, copynumber6_telomeric, copynumber7_telomeric, copynumber8_telomeric, copynumber9_telomeric, copynumber10_telomeric, copynumber11_telomeric, copynumber12_telomeric, copynumber13_telomeric, copynumber14_telomeric, copynumber15_telomeric, changepoint1_telomeric, changepoint2_telomeric, changepoint3_telomeric, changepoint4_telomeric, changepoint5_telomeric, changepoint6_telomeric, changepoint7_telomeric, changepoint8_telomeric, changepoint9_telomeric, changepoint10_telomeric, changepoint11_telomeric, changepoint12_telomeric, or any combination thereof.

At step 108 in FIG. 1, a first trained model is used to analyze the copy number feature values for the plurality of copy number features and identify subgroups of copy number features, and corresponding probabilities that the identified subgroups are represented in the copy number profile of the individual.

In some instances, the first trained model may comprise a trained statistical model. In some instances, the first trained model may comprise a trained machine learning model, e.g., a supervised, semi-supervised, or unsupervised machine learning model. In some instances, the first trained model may be updated based on the one or more copy number signatures identified in the copy number profile of the individual (e.g., the training data set for the model may be updated and used to retrain the first trained model). In some instances, the first trained model may comprise a latent class analysis model or a mixture model.

In some instances, as will be described in more detail below, the first trained model may be trained on copy number feature values calculated for each of the plurality of copy number features based on copy number profiles for a cohort of individuals (e.g., a cohort of patients) diagnosed with the disease. In some instances, the disease may be cancer, e.g., ovarian cancer, prostate cancer, or colorectal cancer.

At step 110 in FIG. 1, a second trained model is used to analyze the probabilities determined for each of one or more copy number feature subgroups to identify one or more copy number signatures represented in the copy number profile of the individual, and to determine their corresponding copy number signature scores.

In some instances, the one or more copy number signatures represented in the copy number profile of the individual may comprise 0, at least 1, at least 2, at least 3, at least 4, at least 5, at least 10, at least 15, at least 20, or more than 20 copy number signatures.

In some instances, the second trained model may comprise, e.g., a non-negative matrix factorization (NMF) model or a machine learning model, such as a supervised, semi-supervised, or unsupervised machine learning model. In some instances, the second trained model may comprise a supervised machine learning model such as an XGBoost model.

In some instances, as will be described in more detail below, the second trained model may be trained on copy number feature subgroups identified in sequencing data for a cohort of individuals (e.g., a cohort of patients) diagnosed with a same disease as that for which the individual whose sample is being processed has been diagnosed. In some instances, the second trained model may be trained on copy number feature subgroups identified in sequencing data for a cohort of individuals comprising individuals diagnosed with a plurality of different diseases. In some instances, the disease with which the individual has been diagnosed may comprise a cancer. In some instances, the plurality of different diseases with which individuals of the cohort used to provide training data have been diagnosed may comprise a plurality of different cancers. In some instances, the plurality of different cancers may comprise, e.g., ovarian cancer, breast cancer, non-small cell lung carcinoma (NSCLC), esophageal cancer, prostate cancer, pancreatic cancer, colorectal cancer, or any combination thereof.

In some instances, as will be described in more detail below, the second trained model may be trained to identify copy number signatures based on the probabilities determined for each of one or more copy number feature subgroups identified in the copy number profiles for the individuals of the cohort, and based on the presence of one or more additional genomic features identified in the sequence read date (e.g., targeted sequence read data) for the individuals of the cohort. In some instances, the one or more additional genomic features may comprise, e.g., one or more short variants, one or more biallelic loss-of-function variants, one or more rearrangements, other pathogenic alterations, or any combination thereof.

At step 112 in FIG. 1, the copy number signature scores determined for the one or more copy number signatures represented in the copy number profile of the individual are compared to one or more corresponding predetermined thresholds (e.g., cutoff thresholds) to identify a dominant copy number signature, or to identify a set of component copy number signatures, for the copy number profile of the individual.

In some instances, the one or more corresponding predetermined thresholds are determined based on an analysis of copy number signature scores for a cohort of individuals (e.g., a cohort of patients) diagnosed with a same disease as that for which the individual whose sample is being processed has been diagnosed. In some instances, the one or more corresponding predetermined thresholds are determined based on an analysis of copy number signature scores for a cohort of individuals (e.g., a cohort of patients) comprising individuals diagnosed with a plurality of different diseases. In some instances, the one or more corresponding predetermined thresholds are determined based on an analysis of data for a cohort of individuals that comprises both copy number signature scores and clinical data. For example, in some instances, the clinical data for the individual (or for the cohort of individuals used to generate training data) may comprise an individual's sex, age, gender, height, weight, clinical history, family history, sample type, tumor stage, tumor grade, or any combination thereof.

In some instances, any of the methods described herein may further comprise confirming a diagnosis of the disease for the individual based on the dominant copy number signature, or the one or more copy number signatures, identified for the individual.

In some instances, any of the methods described herein may further comprise predicting a treatment outcome for the individual based on the dominant copy number signature, or the one or more copy number signatures, identified for the individual.

In some instances, any of the methods described herein may further comprise selecting a treatment for the individual based on the dominant copy number signature or the one or more copy number signatures identified for the individual.

In some instances, any of the methods described herein may further comprise adjusting a treatment dosage for the individual based on the dominant copy number signature or the one or more copy number signatures identified for the individual.

In some instances, any of the methods described herein may further comprise selecting the individual for inclusion in a clinical trial based on the dominant copy number signature or the one or more copy number signatures identified for the individual.

In some instances, any of the methods described herein may further comprise identifying the individual as a candidate for chemotherapy, radiation therapy, or surgery based on the dominant copy number signature or the one or more copy number signatures identified for the individual.

In some instances, any of the methods described herein may further comprise identifying the individual as a candidate for hospice care based on the dominant copy number signature or the one or more copy number signatures identified for the individual.

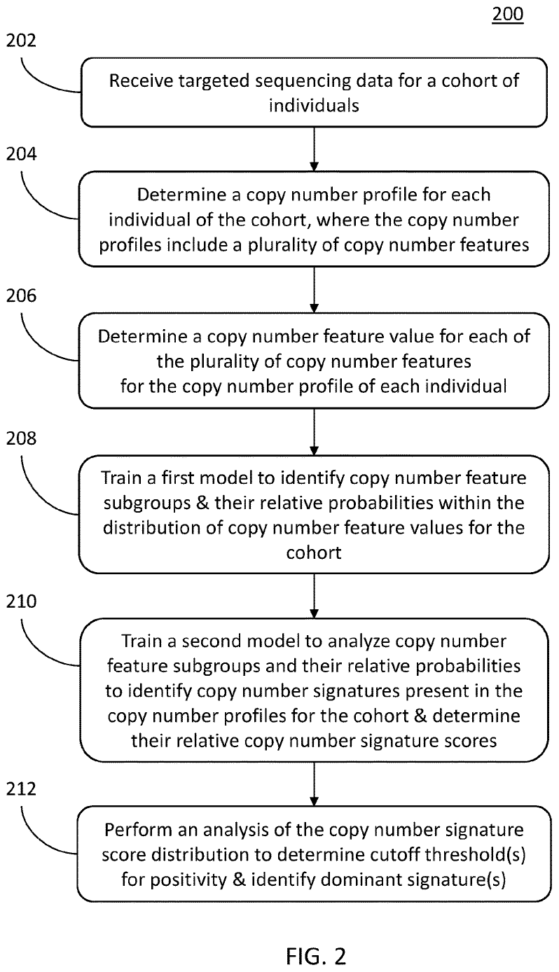

FIG. 2 provides a non-limiting example of a flowchart for a process 200 for training a first model and a second model for use in identifying one or more copy number signatures present in a copy number profile determined from targeted sequencing data for a sample collected from an individual (e.g., a cancer patient).

At step 202 in FIG. 2, a set of targeted sequencing data is received for each of a cohort of individuals (e.g., a cohort of patients), where the targeted sequencing data comprises a plurality of sequence reads obtained by sequencing nucleic acid molecules (e.g., DNA molecules) extracted from samples collected from each individual of the cohort. Methods for collecting samples from individual, extracting nucleic acid molecules therefrom, and sequencing nucleic acid molecules are described elsewhere herein.

In some instances, the samples may be solid biopsy samples (e.g., a tumor samples). In some instances, the samples may be liquid biopsy samples, and may comprise, e.g., whole blood, blood plasma, blood serum, urine, stool, sputum, saliva, or cerebrospinal fluid. In some instances, the samples may be liquid biopsy samples and may comprise cell-free DNA (cfDNA), circulating tumor DNA (ctDNA), or any combination thereof.

In some instances, the generation of targeted sequencing data may comprise use of a next-generation sequencing as described elsewhere herein. As noted above, in some instances, the disclosed methods may be applied to non-targeted sequencing data, e.g., whole genome sequencing data, low pass whole genome sequencing data, whole exome sequencing data, etc.

At step 204 in FIG. 2, a copy number profile is determined for each individual of the cohort, where the copy number profiles can include a plurality of copy number features. Copy number profile determination may be performed using any of a variety of segmentation and/or copy number modeling techniques known to those of skill in the art, e.g., sliding window techniques and hidden Markov Model (HMM) techniques (see, e.g., Wang, et al. (2014), “Copy Number Variation Detection Using Next Generation Sequencing Read Counts”, BMC Bioinformatics 15:109).

In some instances of the disclosed methods, the copy number profile for each individual of the cohort may be determined based on an analysis of off-target sequence reads in the targeted sequence read data for the sample from the individual. In some instances, the copy number profile for the individual may be determined based on an analysis of on-target sequence reads in the targeted sequence read data for the sample from the individual. In some instances, the copy number profile for the individual may be determined based on a combination of on-target and off-target sequence reads in the targeted sequence read data for the sample from the individual.

In some instances of the disclosed methods, the copy number profile for each individual in the cohort is determined based on single nucleotide polymorphisms (SNPs) located in the targeted sequence read data for the sample from the individual. In some instances, the SNPs are located in the off-target sequence reads of the targeted sequence read data set for the individual sample. In some instances, the SNPs are located in the on-target sequence reads of the targeted sequence read data set for the individual sample. In some instances, the SNPs are located in a combination of off-target and on-target sequence reads of the targeted sequence read data set for the individual sample.

As noted above, off-target sequence reads may arise in a targeted sequence read data set for a sample from an individual due to, for example, the inefficiencies of the enrichment strategies used for performing the targeted sequencing. In some instances, the off-target sequence reads may comprise an average sequencing coverage of less than 0.5×, 0.4×, 0.3×, 0.2×, or 0.1×.

In some instances, the copy number profile determined for each individual of the cohort may comprise one or more copy number features (i.e., key attributes of the copy number profile). Examples of copy number features are described in more detail below. In some instances, the copy number profile for an individual of the cohort may comprise at least 1, at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, or more than 80 copy number features.

At step 206 in FIG. 2, a copy number feature value is determined for each of the plurality of copy number features for the copy number profile of each individual in the cohort. As noted above, examples of copy number features to be evaluated include, but are not limited to, the number of breakpoints per 10 Mb of genomic sequence, the number of breakpoints per 25 Mb of genomic sequence, the number of breakpoints per 50 Mb of genomic sequence, the number of breakpoints per 100 Mb of genomic sequence, the number of breakpoints per chromosome arm for each chromosome, the magnitude of a copy number change between any two adjacent copy number segments; the length of each copy number segment; the copy number of each segment; change point copy number, the number of contiguous oscillating copy number chains, the length of oscillating copy number chains, segment minority allele frequency, or any combination thereof.

At step 208 in FIG. 2, a first model is trained to analyze the distribution of copy number feature values for the plurality of copy number profiles represented by the cohort of individuals, and identify subgroups of copy number features and their relative probabilities within the distribution of copy number feature values for the cohort.

In some instances, the first trained model may comprise a trained statistical model. In some instances, the first trained model may comprise a trained machine learning model, e.g., a supervised, semi-supervised, or unsupervised machine learning model. In some instances, the first trained model may comprise a latent class analysis model or a mixture model.

In some instances, the first trained model may be periodically or continuously updated based on an updated training data set that includes the one or more copy number signatures identified in the copy number profile of an individual sample according to the process illustrated in FIG. 1 (e.g., the training data set for the model may be updated and used to retrain the first trained model). In some instances, for example, the adjustable parameters of a machine learning model may be trained using the input data from a training data set and a gradient descent or backward propagation method so that the output value(s) of the model are consistent with the examples included in the training data set.

In some instances, the training data used to train the first trained model may comprise copy number feature values calculated for each of a plurality of copy number features represented in the copy number profiles for the cohort of individuals (e.g., a cohort of patients). In some instances, the training data used to train the first trained model may further comprise the number of copy number feature subgroups identified in the copy number profiles for the individuals or for the cohort of individuals. In some instances, the cohort of individuals may comprise individuals diagnosed with a disease. In some instances, for example, the disease may be cancer, e.g., ovarian cancer, prostate cancer, or colorectal cancer.

At step 210 in FIG. 2, a second model is trained to analyze copy number feature subgroups and their relative probabilities within the distribution of copy number feature values for the cohort to identify copy number signatures present in the copy number profiles for the cohort, and to determine their corresponding copy number signature scores.

In some instances, the one or more copy number signatures represented in the copy number profiles of the individuals in the cohort may comprise at least 1, at least 2, at least 3, at least 4, at least 5, at least 10, at least 15, at least 20, or more than 20 copy number signatures.

In some instances, the second trained model may comprise, e.g., a non-negative matrix factorization (NMF) model or a machine learning model, such as a supervised, semi-supervised, or unsupervised machine learning model. In some instances, the adjustable parameters of the machine learning model may be trained using the input data from a training data set and a gradient descent or backward propagation method so that the output value(s) of the model are consistent with the examples included in the training data set.

In some instances, the second trained model may comprise a supervised machine learning model such as an XGBoost model. XGBoost is an optimized distributed gradient boosting library designed to be highly efficient and flexible that implements machine learning algorithms under the Gradient Boosting framework. XGBoost provides a parallel tree boosting approach to training (using an additive training technique) where, at each iteration, a new tree determines the gradients for the residuals between the target training values (e.g., for copy number signatures and copy number signature scores) and the current predicted values, and the algorithm then conducts a gradient descent-based refinement of the predicted values based on the gradients determined for the residuals.

In some instances, the training data used to train the second trained model may comprise copy number feature subgroups identified in sequencing data for a cohort of individuals (e.g., a cohort of patients) diagnosed with a same disease as that for which the individual whose sample is being processed has been diagnosed. In some instances, the second trained model may be trained on training data comprising copy number feature subgroups identified in sequencing data for a cohort of individuals comprising individuals diagnosed with a plurality of different diseases. In some instances, the training data used to train the second model may further comprise copy number signatures identified in the copy number profiles for the cohort of individuals.

In some instances, the disease with which the individual has been diagnosed (or with which the individuals within the cohort have been diagnosed) may comprise a cancer. In some instances, the plurality of different diseases with which individuals of the cohort used to provide training data have been diagnosed may comprise a plurality of different cancers. In some instances, the plurality of different cancers may comprise, e.g., ovarian cancer, breast cancer, non-small cell lung carcinoma (NSCLC), esophageal cancer, prostate cancer, pancreatic cancer, colorectal cancer, or any combination thereof.

In some instances, the second trained model may be trained to identify copy number signatures based on the probabilities determined for each of one or more copy number feature subgroups identified in the copy number profiles for the cohort of individuals and based on the presence of one or more additional genomic features identified in the sequence read data (e.g., targeted sequence read data) for the individuals of the cohort. In some instances, the one or more additional genomic features may comprise, e.g., one or more short variants, one or more biallelic loss-of-function variants, one or more rearrangements, other pathogenic alterations, or any combination thereof.

At step 212 in FIG. 2, an analysis of the copy number signature score distribution is performed to calculate cutoff threshold(s) for determining a positive presence of a given copy number signature in a given copy number profile, and to identify dominant copy number signature(s). Cutoff thresholds may be calculated, for example, using an elbow method to discriminate between baseline noise and signal, a signal-to-noise analysis, or other statistical approaches such as random sampling/bootstrapping.

### Copy Number Signatures as Biomarkers

In some instances, the methods described herein may be used to confirm a diagnosis of disease for an individual based on the dominant copy number signature, or the one or more copy number signatures, identified for the individual.

In some instances, the methods described herein may be used to predict a treatment outcome for an individual based on the dominant copy number signature, or the one or more copy number signatures, identified for the individual.

In some instances, the methods described herein may be used to select a treatment for an individual based on the dominant copy number signature or the one or more copy number signatures identified for the individual.

For example, in some embodiments, the methods described herein may be used to identify an individual that may benefit from treatment with a poly (ADP-ribose) polymerase inhibitor (PARPi). In some embodiments, the methods of the present disclosure comprise identifying a dominant copy number signature or one or more copy number signatures that are components of a copy number profile for the individual, in which the dominant copy number signature, one or more component copy number profile, and/or copy number profile comprise a homologous recombination deficient (HRD) signature. The examples provided infra demonstrate that the methods of the present disclosure allow for improved detection of an HRD signature in a sample, and that such HRD signatures have predictive value for patients that may benefit from PARPi treatment. Any of the methods of the present disclosure may find use in identifying an individual that may benefit from PARPi treatment. In some embodiments, the individual has ovarian or prostate cancer. In some embodiments, the individual is a human.

In some embodiments, the sample comprising an HRD signature as set forth herein lacks a loss-of-function mutation in BRCA1 and/or BRCA2 gene(s). In some embodiments, the sample comprising an HRD signature as set forth herein lacks a loss-of-function mutation in BRCA1 and BRCA2 genes, e.g., is BRCAwt. In some embodiments, the sample comprising an HRD signature as set forth herein lacks a loss-of-function mutation in BRCA1, BRCA2, ATM, BARD1, BRIP1, CDK12, CHEK1, CHEK2, FANCL, PALB2, RAD51B, RAD51C, RAD51D, and/or RAD54L gene(s). In some embodiments, the sample comprising an HRD signature as set forth herein lacks a loss-of-function mutation in BRCA1, BRCA2, ATM, BARD1, BRIP1, CDK12, CHEK1, CHEK2, FANCL, PALB2, RAD51B, RAD51C, RAD51D, and RAD54L genes, e.g., is HRRwt. Whereas current patient selection strategies primarily rely on the presence of germline and/or tumor alterations in BRCA1 and BRCA2, the present disclosure demonstrates that the HRD signature identified a BRCA1/2 wildtype population enriched for biallelic alterations in other homologous recombination repair (HRR) genes and in some cases lacking any HRR alteration, suggesting that using the HRD signature allows for detection of a population not currently eligible for PARPi treatment in many indications.

In some embodiments, the sample comprising an HRD signature as set forth herein lacks a loss-of-function mutation in BRCA1 and/or BRCA2 gene(s) and comprises biallelic loss-of-function mutations in one or more of BARD1, PALB2, RAD51D, and RAD51C gene(s). In some embodiments, the sample comprising an HRD signature as set forth herein lacks a loss-of-function mutation in BRCA1and BRCA2 genes (e.g., is BRCAwt) and comprises biallelic loss-of-function mutations in one or more of BARD1, PALB2, RAD51D, and RAD51C gene(s). The present disclosure demonstrates that the HRD signature identified a BRCAwt population strongly associated with biallelic alterations in BARD1, PALB2, RAD51D, and/or RAD51C.

In some embodiments, an HRD signature of the present disclosure comprises a plurality of low copy number segments. In some embodiments, the HRD signature comprises heterozygous deletion(s), high genome-wide loss of heterozygosity (gLOH-high), and/or high genomic instability score (GIS). In some embodiments, an HRD signature of the present disclosure comprises a plurality of high copy number amplifications, e.g., amplifications of more than 70 copies gained. In some embodiments, e.g., in gynecologic cancer such as ovarian or breast cancer, the HRD signature further comprises heterozygous deletion(s) (e.g., segment copy numbers at half of ploidy) and/or duplication(s) (e.g., ploidy+1 in diploid samples). In some embodiments, e.g., in gynecologic cancer such as ovarian or breast cancer, the HRD signature further comprises heterozygous deletion(s) (e.g., segment copy numbers at half of ploidy) and/or duplication(s) (e.g., ploidy+1 in diploid samples), and is characterized by gLOH-high status. In some embodiments, gLOH-high refers to greater than or equal to 16% of the genome under LOH. In some embodiments, e.g., in prostate cancer, the HRD signature further comprises heterozygous deletion(s) (e.g., segment copy numbers at half of ploidy) without duplication(s) (e.g., segments at ploidy, or segment copy number 1.6-2.2 for diploid samples), e.g., copy neutral loss of heterozygosity.

In some embodiments, the methods of the present disclosure further comprise administering an effective amount of a PARPi to the individual. In some embodiments, PARPi inhibits PARP1 and PARP2. In some embodiments, PARPi inhibits PARP1, PARP2, and PARP3. PARPi's are known in the art, and non-limiting examples of PARPi's are described below.

In some embodiments, the PARPi comprises olaparib (also known as AZD2281, MK-7339, KU0059436, or LYNPARZA®; AstraZeneca/Merck). The structure of olaparib is provided below.

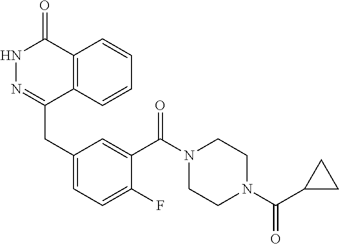 [SMILES: O=C(c1cc(Cc2n[nH]c(=O)c3ccccc23)ccc1F)N1CCN(C(=O)C2CC2)CC1]

In some embodiments, the PARPi comprises rucaparib (also known as AG014699, PF-01367338, or RUBRACA®; Clovis Oncology). The structure of rucaparib is provided below.

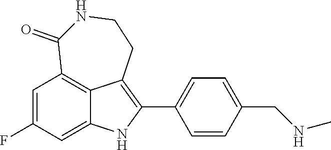 [SMILES: CNCc1ccc(-c2[nH]c3cc(F)cc4c3c2CCNC4=O)cc1]

In some embodiments, the PARPi comprises niraparib (also known as MK-4827 or ZEJULA®; GlaxoSmithKline). The structure of niraparib is provided below.

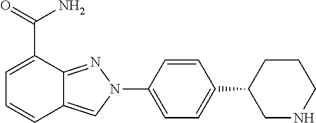 [SMILES: NC(=O)c1cccc2cn(-c3ccc([C@@H]4CCCNC4)cc3)nc12]

In some embodiments, the PARPi comprises talazoparib (also known as BMN-673 or TALZENNA®; Pfizer). The structure of talazoparib is provided below.

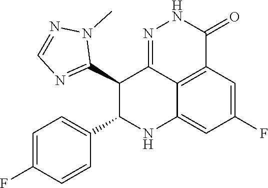 [SMILES: Cn1ncnc1[C@H]1c2n[nH]c(=O)c3cc(F)cc(c23)N[C@@H]1c1ccc(F)cc1]

In some embodiments, the PARPi comprises veliparib (also known as ABT-888). The structure of veliparib is provided below.

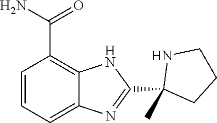 [SMILES: C[C@]1(c2nc3cccc(C(N)=O)c3[nH]2)CCCN1]

In some instances, the methods described herein may be used to adjust a treatment dosage for an individual based on the dominant copy number signature or the one or more copy number signatures identified for the individual.

In some instances, the methods described herein may be used to selecting an individual for inclusion in a clinical trial based on the dominant copy number signature or the one or more copy number signatures identified for the individual.

In some instances, the methods described herein may be used to identify an individual as a candidate for chemotherapy, radiation therapy, or surgery based on the dominant copy number signature or the one or more copy number signatures identified for the individual.

In some instances, the methods described herein may be used to identify an individual as a candidate for hospice care based on the dominant copy number signature or the one or more copy number signatures identified for the individual.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for identifying copy number signatures may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for identifying copy number signatures may be used to select a subject (e.g., a patient) for a clinical trial based on the dominant copy number signature, or set of copy number signature components, identified in the subject's copy number profile. In some instances, patient selection for clinical trials based on, e.g., identification of copy number signatures, may accelerate the development of targeted therapies and improve the healthcare outcomes for treatment decisions.

In some instances, the disclosed methods for identifying copy number signatures may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for identifying copy number signatures may be used in treating a disease (e.g., a cancer) in a subject. For example, in response to identifying a dominant copy number signature, or a set of one or more component copy number signatures, using any of the methods disclosed herein, an effective amount of an anti-cancer therapy or anti-cancer treatment may be administered to the subject.

In some instances, the disclosed methods for identifying copy number signatures may be used for monitoring disease progression or recurrence (e.g., cancer or tumor progression or recurrence) in a subject. For example, in some instances, the methods may be used to identify a dominant copy number signature, or a set of component copy number signatures, in a first sample obtained from the subject at a first time point, and used to identify a dominant copy number signature, or a set of component copy number signatures, in a second sample obtained from the subject at a second time point, where comparison of the first determination of copy number signatures and the second determination of copy number signatures allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the dominant copy number signature or set of component copy number signatures.

In some instances, the dominant copy number signature, or set of component copy number signatures, identified using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for identifying copy number signatures may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for identifying copy number signatures as part of a genomic profiling process (or inclusion of the output from the disclosed methods for identifying copy number signatures as part of the genomic profile of the subject) can improve the validity of, e.g., disease detection calls and treatment decisions, made on the basis of the genomic profile by, for example, independently confirming the presence of a dominant copy number signature or set of component copy number signatures in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs).

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule.

For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S.L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions—deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B.L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C.A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S.Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for identifying copy number signatures in a sample from a subject. The systems may comprise, e.g., one or more processors, and a memory unit communicatively coupled to the one or more processors and configured to store instructions that, when executed by the one or more processors, cause the system to: receive targeted sequence read data for a sample derived from an individual diagnosed with or suspected of having a disease; determine a copy number profile having a plurality of copy number features for the individual based on the targeted sequence read data for the sample; determine a copy number feature value for each of the plurality of copy number features; perform an analysis of the copy number feature values for each of a plurality of copy number features using a first trained model to determine a probability that each of one or more copy number feature subgroups is represented in the copy number profile for the sample; perform an analysis of the probabilities determined for each of the one or more copy number feature subgroups using a second trained model to identify one or more copy number signatures represented in the copy number profile for the individual, and to determine one or more corresponding copy number signature scores; compare the copy number signature scores for the one or more identified copy number signatures to one or more corresponding predetermined thresholds; and identify, based on the comparison, a dominant copy number signature for the individual; or identify, based on the comparison, one or more copy number signatures that are components of the copy number profile for the individual.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for identifying copy number signatures in sequencing data obtained from any of a variety of samples as described elsewhere herein (e.g., a tissue sample, biopsy sample, hematological sample, or liquid biopsy sample derived from the subject).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the identification of a dominant copy number signature, or a set of component copy number signatures, may be used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

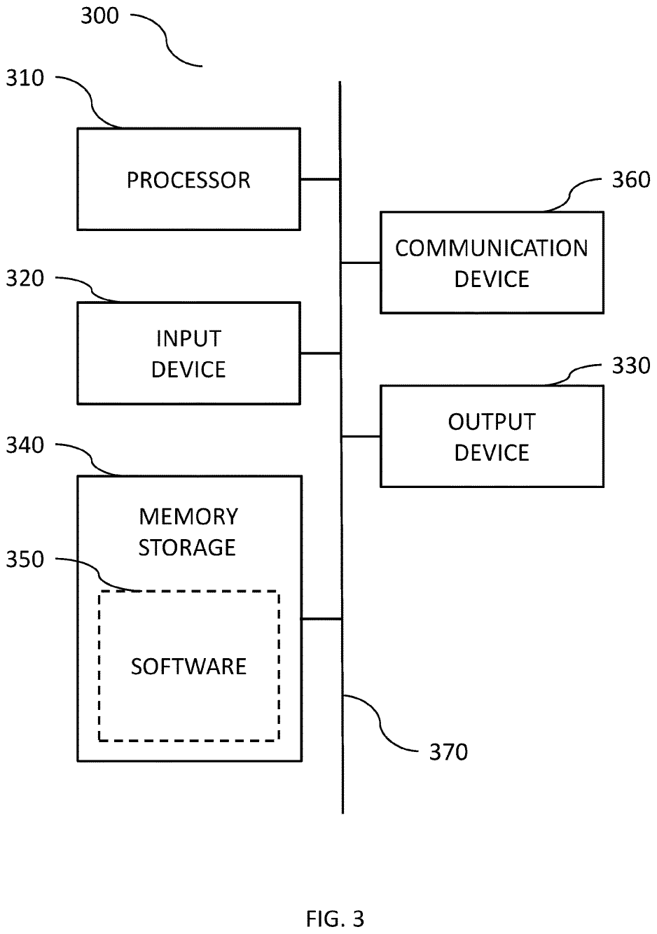

FIG. 3 illustrates an example of a computing device or system in accordance with one embodiment. Device 300 can be a host computer connected to a network. Device 300 can be a client computer or a server. As shown in FIG. 3, device 300 can be any suitable type of microprocessor-based device, such as a personal computer, workstation, server or handheld computing device (portable electronic device) such as a phone or tablet. The device can include, for example, one or more processor(s) 310, input devices 320, output devices 330, memory or storage devices 340, communication devices 360, and nucleic acid sequencers 370. Software 350 residing in memory or storage device 340 may comprise, e.g., an operating system as well as software for executing the methods described herein. Input device 320 and output device 330 can generally correspond to those described herein, and can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage (e.g., an electrical, magnetic or optical memory including a RAM (volatile and non-volatile), cache, hard drive, or removable storage disk). Communication device 360 can include any suitable device capable of transmitting and receiving signals over a network, such as a network interface chip or device. The components of the computer can be connected in any suitable manner, such as via a wired media (e.g., a physical system bus 380, Ethernet connection, or any other wire transfer technology) or wirelessly (e.g., Bluetooth®, Wi-Fi®, or any other wireless technology).

Software module 350, which can be stored as executable instructions in storage 340 and executed by processor(s) 310, can include, for example, an operating system and/or the processes that embody the functionality of the methods of the present disclosure (e.g., as embodied in the devices as described herein).

Software module 350 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 340, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

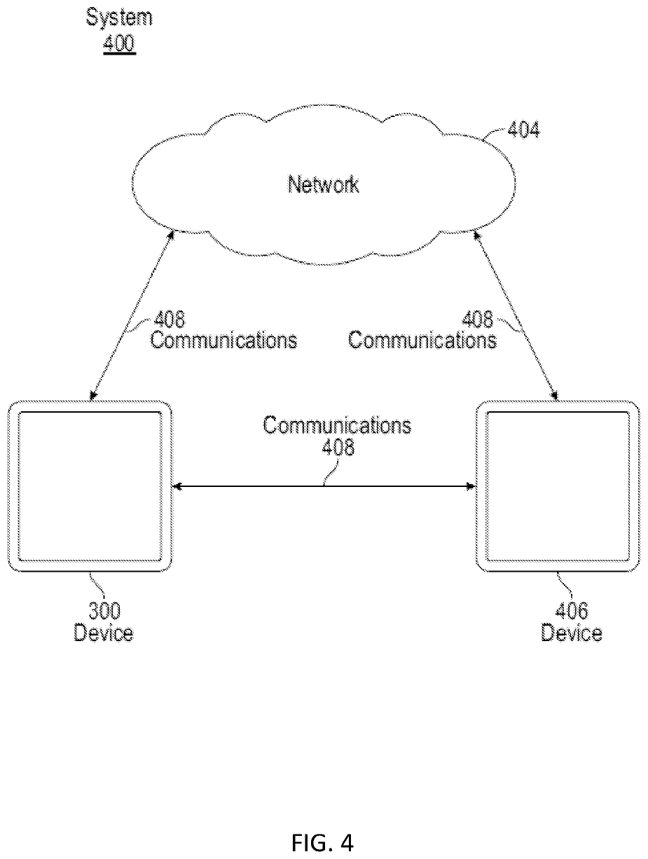

Device 300 may be connected to a network (e.g., network 404, as shown in FIG. 4 and/or as described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can be implemented using any operating system, e.g., an operating system suitable for operating on the network. Software module 350 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 310.

Device 300 can further include a sequencer 370, which can be any suitable nucleic acid sequencing instrument.

FIG. 4 illustrates an example of a computing system in accordance with one embodiment. In system 400, device 300 (e.g., as described above and illustrated in FIG. 3) is connected to network 404, which is also connected to device 406. In some embodiments, device 406 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 300 and 406 may communicate, e.g., using suitable communication interfaces via network 404, such as a Local Area Network (LAN), Virtual Private Network (VPN), or the Internet. In some embodiments, network 404 can be, for example, the Internet, an intranet, a virtual private network, a cloud network, a wired network, or a wireless network. Devices 300 and 406 may communicate, in part or in whole, via wireless or hardwired communications, such as Ethernet, IEEE 802.11b wireless, or the like. Additionally, devices 300 and 406 may communicate, e.g., using suitable communication interfaces, via a second network, such as a mobile/cellular network. Communication between devices 300 and 406 may further include or communicate with various servers such as a mail server, mobile server, media server, telephone server, and the like. In some embodiments, Devices 300 and 406 can communicate directly (instead of, or in addition to, communicating via network 404), e.g., via wireless or hardwired communications, such as Ethernet, IEEE 802.11b wireless, or the like. In some embodiments, devices 300 and 406 communicate via communications 408, which can be a direct connection or can occur via a network (e.g., network 404).

One or all of devices 300 and 406 generally include logic (e.g., http web server logic) or are programmed to format data, accessed from local or remote databases or other sources of data and content, for providing and/or receiving information via network 404 according to various examples described herein.

### EXAMPLES

### Example 1—Pan-Cancer Analysis of Copy Number Signatures Identifies Broad Prevalence and Utility in Predicting Response to PARP Inhibitors

This section provides a non-limiting example of how copy number signatures can be used to reveal the molecular states of various cancers and provide prognostic value for identifying treatments. The landscapes of 10 copy number signatures found in a pan-cancer cohort of 259,415 samples were analyzed, identifying associations with genomic alterations and clinical characteristics. Copy number signatures were seen at a high prevalence across many cancer types, and all 10 signatures were present in 41/46 of cancer types analyzed, highlighting their broad relevance.

This work demonstrates that the features underlying copy number signatures can be utilized for a machine learning based homologous recombination deficiency caller (HRDsig) that has predictive value, e.g., for PARP inhibitor benefit in real-world datasets for ovarian and prostate cancer. These results highlight that characterizing copy number signatures from targeted capture panel genomic profiling across many cancer types may have utility as a clinical decision-making tool.

### BACKGROUND

Cancers dominated by copy number changes have a propensity to overcome therapy, metastasize and have poor prognosis (see, e.g., Bakhoum S F et al. (2018) Nature 553:467-472; Bielski C M et al. (2018) Nat Genet 50(8):1189-1195; Carter S L et al. (2012) Nat Biotechnol 30(5):413-421; Taylor A M et al. (2018) Cancer Cell 33(4):676-689.e673). Thus, there is an urgent need to identify targetable weaknesses in these tumors (Ciriello G et al. (2013) Nat Genet 45(10):1127-1133). Copy number signatures, generated by extracting patterns in aberrant copy numbers from the whole genome, identify complex biological processes relevant to tumor biology (Macintyre G et al. (2018) Nat Genet 50(9):1262-1270; Wang S et al. (2021) PLoS Genet 17(5):e1009557; Pladsen A V et al. Commun Biol 2020, 3(1):153; Maclachlan K H et al. Nat Commun 2021, 12(1):5172; Guan B et al. Front Cell Dev Biol 2021, 9:713499; Steele C D et al. Signatures of copy number alterations in human cancer. bioArxiv 2021). Previous studies have demonstrated possible prognostic and predictive value of copy number signatures in certain therapeutic contexts (Macintyre G et al. (2018) Nat Genet 50(9):1262-1270; Pladsen A V et al. Commun Biol 2020, 3(1):153; Maclachlan K H et al. Nat Commun 2021, 12(1):5172; Guan B et al. Front Cell Dev Biol 2021, 9:713499). In breast, Pladsen and colleagues characterized copy number features to form an independent prognostic predictor for immune checkpoint inhibitor response. In high grade serous ovarian cancer (HGSOC), Macintyre and colleagues predicted worse outcomes in their breakage-fusion-bridge signature and better outcomes in their BRCA1/2-related homologous recombination deficiency (HRD) signature (Macintyre G et al. (2018) Nat Genet 50(9):1262-1270).

One area where genomic signatures have demonstrated value is in the PARP inhibitor (PARPi) space (Moore K N et al. Lancet Oncol 2019, 20(5):636-648; Coleman R L et al. Lancet 2017, 390(10106):1949-1961). Selecting the right patients for PARPi therapy remains a challenge as genomic mutations may not capture the full population of patients that can potentially benefit therapeutically. For example, BRCA1promoter hypermethylation has been reported to cause an HRD phenotype in ovarian and breast cancers but isn't captured with DNA mutation calling (Ruscito I et al. Eur J Cancer 2014, 50(12):2090-2098 Yamashita N et al. Clin Breast Cancer 2015, 15(6):498-504). Conversely, monoallelic alterations in BRCA1 or BRCA2 may not cause an HRD phenotype (Sokol E S et al. JCO Precis Oncol 2020, 4:442-465) and associations with HRD for genes beyond BRCA1/2 are mixed (Westphalen B et al. Clin Cancer Res 2022 28(7):1412-1421). In ovarian cancer, genomic-scar based measures for HRD have been developed that look for a genomic ‘BRCAness’ signature. Two diagnostics are currently approved: genome-wide loss of heterozygosity (gLOH) and genomic instability score (GIS); however, it is unclear how broadly these signatures may be applied beyond ovarian cancer. In prostate cancer, a basket of 14 homologous recombination repair (HRR) genes is used as a companion diagnostic for PARPi selection; however, many of the eligible patients will not respond (de Bono J et al. N Engl J Med 2020, 382(22):2091-2102; Taza F et al. Nat Biotechnol 2013, 31(11):1023-1031).

This example describes the landscape of copy number signatures in a large real-world genomic dataset obtained from comprehensive genomic profiling (CGP) using a hybrid capture targeted gene panel assay. 259,415 samples across 46 tumor types were analyzed for copy number aberrations using feature quantification and signature extraction methods published by Macintyre and colleagues (Macintyre G et al. (2018) Nat Genet 50(9):1262-1270). From this pan-cancer cohort, 10 biologically informative copy number signatures were characterized.

This example demonstrates that the underlying features for copy number signatures can be utilized for a machine learning based caller (such as a HRD caller, e.g., HRDsig) which has predictive value for PARPi benefit in real-world datasets of ovarian and prostate cancer. This highlights the benefit of quantifying copy number aberrations in the cancer genome to characterize copy number signatures across many cancer types and also the utility for clinical decision making.

### Materials and Methods

### Dataset

Tumor samples were sequenced (August 2014 to April 2021) by hybrid capture-based comprehensive genomic profiling as part of routine clinical care (Frampton G M et al. Nat Biotechnol 2013, 31(11):1023-1031). Sequencing of liquid biopsy ctDNA was performed on ≥20 ng of ctDNA extracted from blood plasma to create adapter sequencing libraries before hybrid capture and sample-multiplexed sequencing to a median unique exon coverage depth of >6,000× for up to 324 genes (Woodhouse R et al. PLoS One 2020, 15(9):e0237802). Results were analyzed for base substitutions, short insertions and deletions (indels), copy number alterations, and rearrangements. Testing was performed in a CLIA certified/CAP-accredited laboratory.

### Clinico-Genomic Database and PARPi Real World Outcomes

This study used the nationwide (US-based) de-identified Flatiron Health-Foundation Medicine ovarian and metastatic prostate clinico-genomic databases. The de-identified data originated from approximately 280 US cancer clinics (~800 sites of care). Retrospective longitudinal clinical data were derived from electronic health record (EHR) data, comprising patient-level structured and unstructured data, curated via technology-enabled abstraction, and were linked to genomic data derived from comprehensive genomic profiling (CGP) tests in the databases by de-identified, deterministic matching (Singal G et al. JAMA 2019, 321(14):1391-1399). The study included 4,292 and 3,551 patients diagnosed with ovarian and metastatic prostate cancer, respectively, who received care within the network between January 2011-September 2021. Clinical characteristics and oral therapy usage were obtained via technology-enabled abstraction of clinical notes and radiology/pathology reports and linked to CGP data (Singal G et al. JAMA 2019, 321(14):1391-1399).

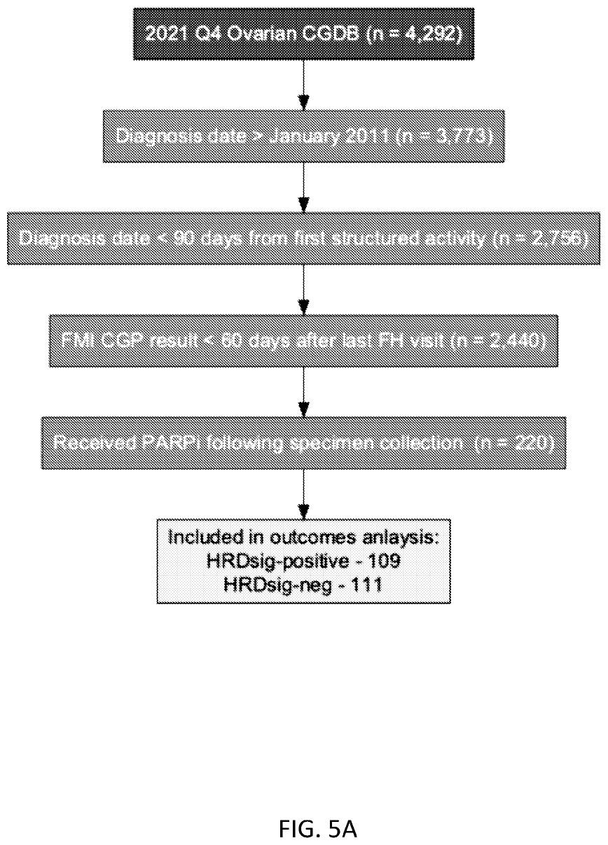

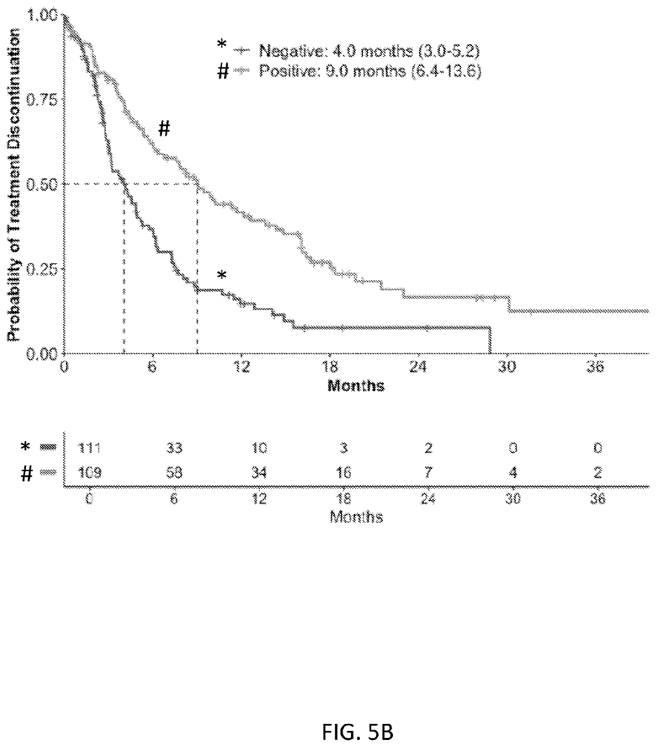

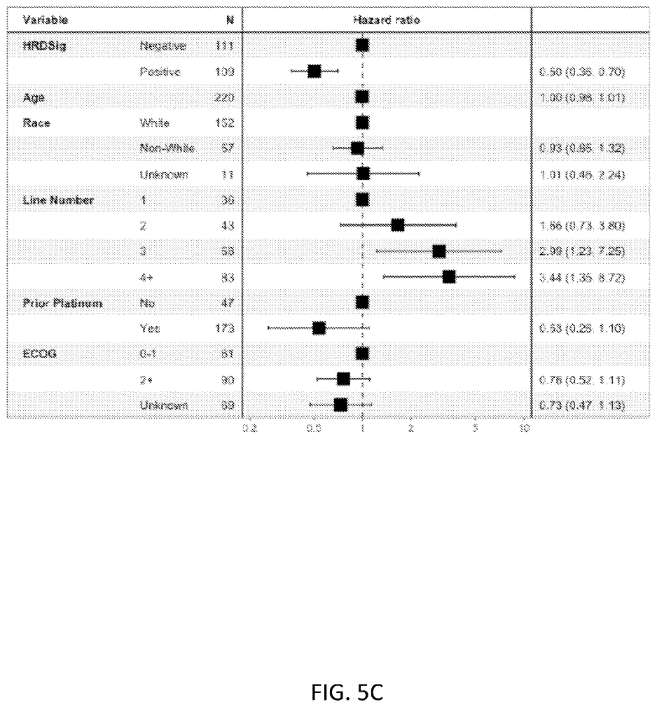

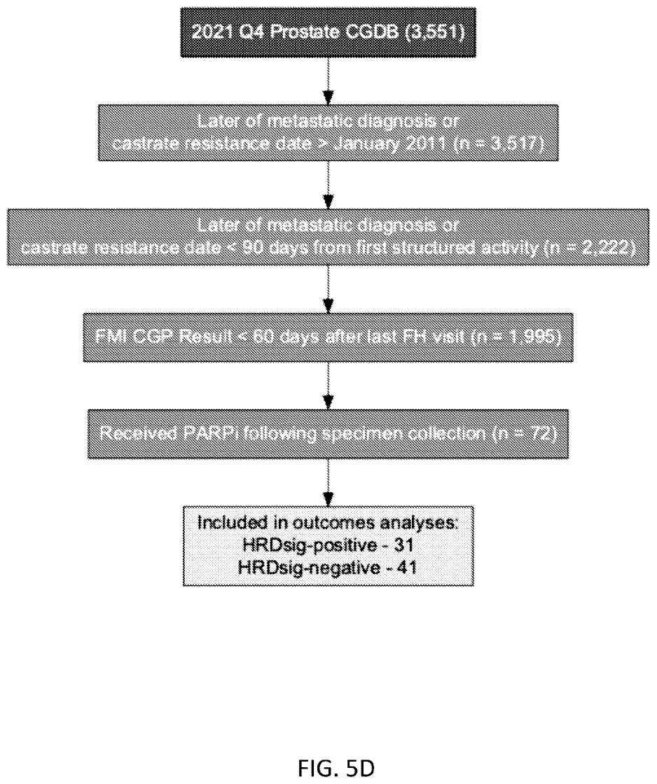

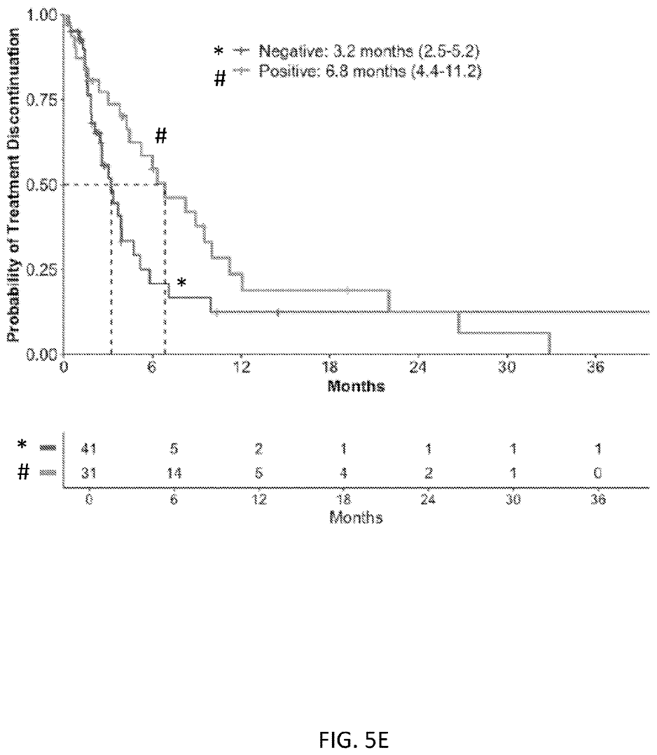

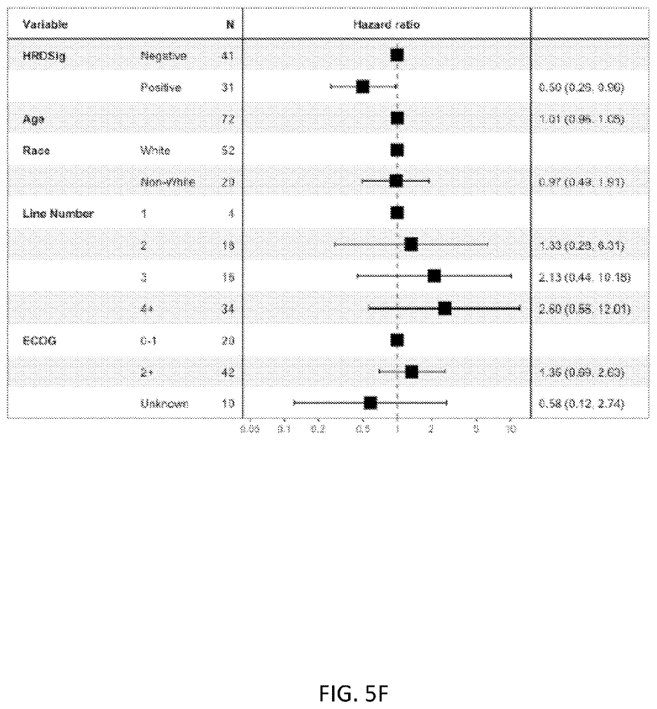

Additional inclusion criteria were applied to select a cohort for outcomes analysis: 1) an ovarian carcinoma diagnosis (ovarian) or mCRPC diagnosis (prostate) date in 2011 or later 2) a diagnosis date no greater than 90 days following first structured activity 3) relevant CGP result no more than 60 days following last visit to network 4) tissue CGP with collection date prior to initiation of PARPi. This left 220 and 72 PARPi treated patients with ovarian or prostate cancer, respectively (FIGS. 5A & 5D).

Median time to treatment discontinuation (TTD) was estimated with Kaplan-Meier analysis and adjusted (age at PARPi initiation, line number, race, ECOG and prior platinum exposure [ovarian only]) hazard ratios (HR) from Cox proportional hazards models. PARPi treatment was considered discontinued if a subsequent line of systemic therapy which did not include PARPi was documented, structured data was present 90 days or more following the last documented drug episode date, or if the patient died within 90 days of the most recent drug episode date. All other patients were censored at the last abstracted PARPi date. For prostate patients, therapy line numbers were indexed from the date of castrate resistant disease. Therapies received prior to this date were not counted towards cumulative line count.

Calculation of gLOH and GIS

Fraction focal genome-wide loss of heterozygosity (gLOH) was calculated by quantifying loss of heterozygosity across the genome but excluding whole chromosome arm losses (>90% arm loss), a method that was described and validated in the ARIEL2 and ARIEL3 clinical trials for ovarian cancer (Coleman R L et al. Lancet 2017, 390(10106):1949-1961; Swisher E M et al. Lancet Oncol 2017, 18(1):75-87). Genome instability scores were calculated from segmented copy number profiles and represented a sum of qualifying LOH segments (Abkevich V et al. Br J Cancer 2012, 107(10):1776-1782), TAI events (Birkbak N J et al. Cancer Discov 2012, 2(4):366-375), and LST adjusted for ploidy (Popova T et al. Cancer Res 2012, 72(21):5454-5462) by subtracting 15.5*ploidy (Timms K M et al. Breast Cancer Res 2014, 16(6):475).

### XGBoost Machine Learning Model to Predict BRCAness

Copy number features and indel features were included in constructing the extreme gradient boosting (XGB) machine learning model using the caret package in R (Kuhn M: caret: Classification and Regression Training. In., R package version 6.0-90. edn; 2021: https://CRAN.R-project.org/package=caret). Biallelic BRCA1/2 and homologous recombination repair wildtype (HRRwt; wildtype for BRCA1, BRCA2, ATM, BARD1, BRIP1, CDK12, CHEK1, CHEK2, FANCL, PALB2, RAD51B, RAD51C, RAD51D, RAD54L alterations) were defined as true positive and true negative training labels. The data was divided into training and testing datasets (70:30). For feature selection, all the features were initially used to fit a model in training dataset. The features were then ranked by their importance based on their performance gain. The optimal number of features was selected until the ROC-AUC plateaued in the training dataset. The optimal model was determined using grid search over the hyper-parameter space with 4 repeated 10-fold cross-validation and the performances are evaluated by ROC-AUC. During development it was noted that genomic scarring of the prostate is different than other tumor types, so two XGB models were developed, one applied to pan-cancer and the other applied to prostate only. Both XGB models (pan-cancer and prostate-specific) were trained in the same process.

### Mixture Modeling

To train a copy number signature model, high quality samples were selected that passed a computational and manual QC curation process (Frampton G M et al. Nat Biotechnol 2013, 31(11):1023-1031); they had a computational purity >=15%, they did not contain significant contamination, and they were not a confirmed transplant. TP53 mutated ovarian carcinoma tissue biopsy samples were selected that had been assayed as part of regular clinical care. Absolute copy number of segments was normalized by the mean ploidy of sample. This yielded a more robust measurement of the copy number changes seen in samples. Biologically informative heuristic models for breakpoint features were used to capture different densities of breakpoints, looking at breakpoints per 10MB, 25 MB, 50 MB, and 100 MB. This is a deviation from Macintyre and colleagues but was more robust and didn't overfit on sparse breakpoint data and was biologically informed which is an approach used in Wang et al (PLoS Genet 2021, 17(5):e1009557).

### Generating Signatures

To generate pan-cancer copy number signatures 2000 samples were selected from key diseases: ovary, breast, lung non-small cell lung carcinoma (NSCLC), esophageal, prostate, and pancreatic cancer (Ciriello G et al. (2013) Nat Genet 45(10):1127-1133; Macintyre G et al. (2018) Nat Genet 50(9):1262-1270). To ensure rarer diseases were included in the testing set, up to 200 samples were added from these tumor types. When <200 samples were available in a disease group all samples were added. This cohort was split into 75% training and 25% testing. Rank for non-negative matrix factorization was estimated by picking a rank between 2 and 15 where the sparseness in the basis matrix was higher in a randomized dataset than in the original dataset (Macintyre G et al. (2018) Nat Genet 50(9):1262-1270). The dispersion and cophenetic coefficients were also calculated between consecutive runs to evaluate the different numbers of ranks (Brunet J P et al. Proc Natl Acad Sci USA 2004, 101(12):4164-4169). Once a rank was selected, Nonnegative Double Singular Value Decomposition (NNSVD) was used to seed the NMF algorithm using Nimfa (v1.2.3) (Žitnik M, Zupan B: NIMFA: A Python Library for Nonnegative Matrix Factorization. Journal of Machine Learning Research 2012). It was found that most samples had very high exposure to the low aneuploidy signature and had low exposure to the other 9 signatures. In the other 9 signatures, most samples had very little exposure to the signature, but a few had very high exposure. An elbow method was used to derive a cutoff for these high exposure population in each signature. If samples were above this cutoff for exposure to signatures 2 through 9, they were called positive for that signature. If samples did not have a signature with exposure greater than its cutoff in any signature in signatures 2 through 9 and was above the elbow cutoff for the low aneuploidy signature (like most samples), it was called positive for the low aneuploidy signature.

Association of Copy Number Signatures with Other Features

For comparisons of categorical variables, the Fisher's exact test was used. For comparisons of continuous variables, the Mann-Whitney U test was used. To control for disease patterns impacting results, disease balanced cohorts were used when looking for associations with features and copy number signatures by down-sampling tumor samples used in the analysis so that the number of samples in each disease were the same on either side of the test. For Fisher's exact tests where multiple hypotheses were tested, Benjamini-Hochberg procedure was used to reduce false discovery rate.

### Tumor Fraction

Tumor fraction was estimated using a measure of tumor aneuploidy that incorporates observed deviations in coverage across the genome for a given sample. Calculated values for this metric were calibrated against a training set based on samples with well-defined tumor fractions to generate an estimate of the tumor fraction (Li M et al. Cancer Res 2021, 81 (13_Supplement): 2231).

### Telomeric Content

The telomeric content of samples was determined using TelomereHunter 1.1.0 (Feuerbach L et al. BMC Bioinformatics 2019, 20(1):272). This software tool was run using the default parameters and a repeat threshold set to 7 for 49 bp paired-end reads. TelomereHunter extracts telomeric reads that contain seven instances of the four most common telomeric repeat types (TTAGGG, TCAGGG, TGAGGG, and TTGGGG) and determines the telomeric content by normalizing the telomere read count by all reads in the sample with a GC content of 48-52%.

### Results

### Landscape of Copy Number Signatures in a Pan-Cancer Cohort

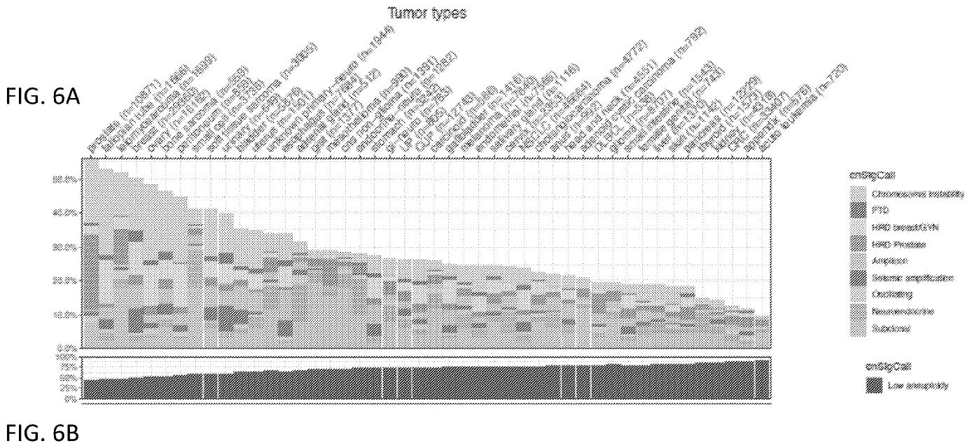

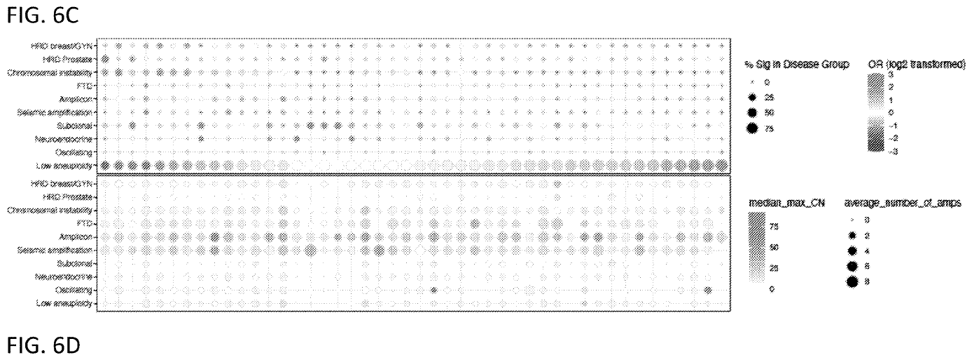

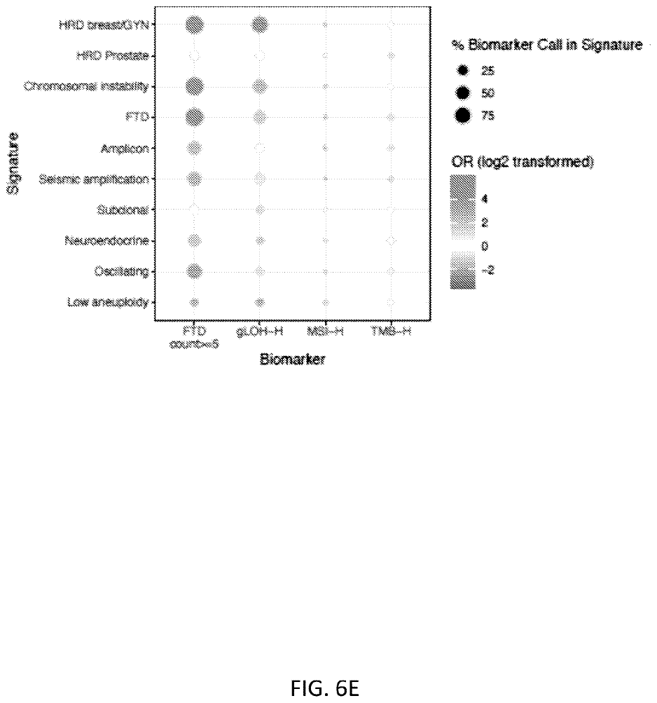

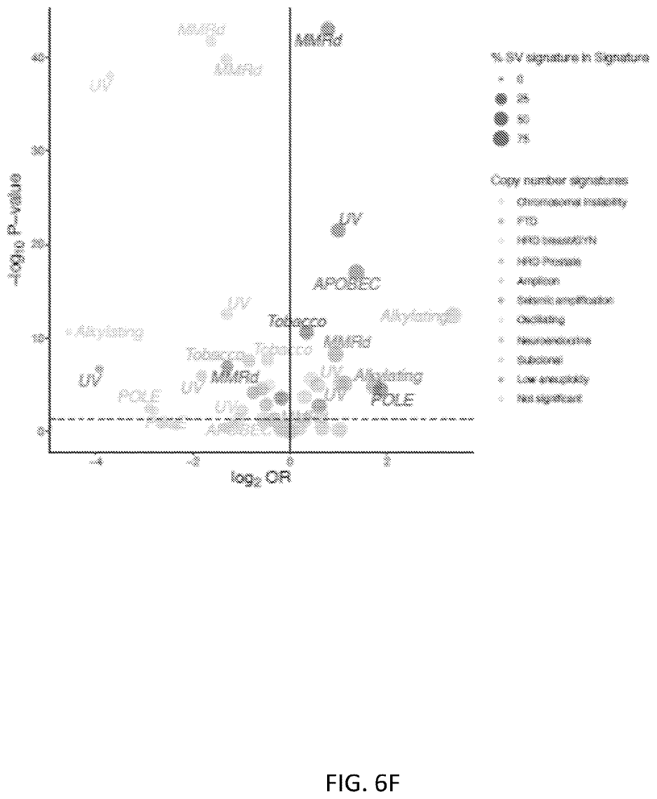

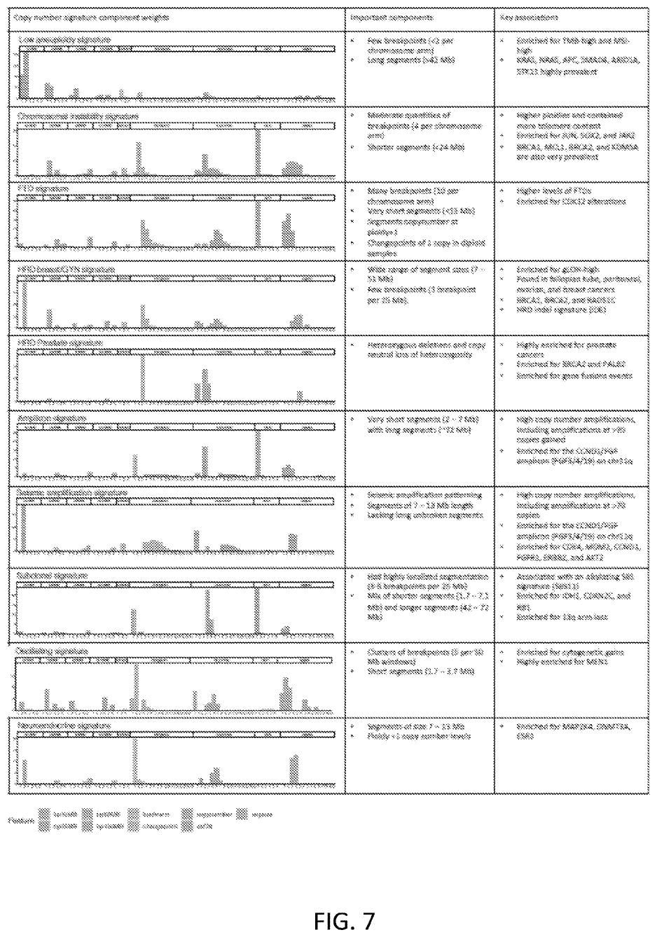

Key genomic patterns were quantified in a pan-cancer cohort of 259,415 samples spanning 46 diseases (FIG. 6A). The following features were extracted from each sample copy number profile: the number of breakpoints (per 10 Mb, 25 Mb, 50 Mb, 100 Mb, and chromosome arm); the magnitude of copy number changes between two adjacent segments; the length of segments; the copy number of segments; and the count of contiguous oscillating copy number chains. Using a mixture modeling approach, 76 whole genome copy number features were generated. By clustering these data using non-negative matrix factorization 10 copy number signatures were identified (FIGS. 7 & 8; Macintyre G et al. (2018) Nat Genet 50(9):1262-1270; Brunet J P et al. Proc Natl Acad Sci USA 2004, 101(12):4164-4169). Based on the copy number signature patterns and sample characteristics, an etiology was proposed for each signature.

A low aneuploidy signature was identified and was dominant in 70.5% of samples (FIG. 6B). Unlike all other copy number signatures, this signature was defined by the absence of copy number aberrations, such as few breakpoints (<2 per chromosome arm) and long segments (>42 Mb) (Fig S1 & S2). The low aneuploidy signature was most prevalent in acute leukemias (90%) and appendiceal cancers (88%). This signature was also enriched for tumors that are associated with well characterized hypermutation phenotypes rather than chromosomal instability, including colorectal cancers (87%), skin cancers (82%) and diffuse large B-cell lymphoma (80%) (FIGS. 6B & 6C). Samples in this signature were also enriched for high tumor mutational burden (>=20 mutations per Mb; TMBhigh) and high microsatellite instability (MSI-high) (FIG. 6E), mismatch repair deficiency (MMRd), ultraviolet deamination (UV), and defunct DNA polymerase epsilon (POLE) mutational processes (FIG. 6F). Diseases with low rates of low aneuploidy signature included prostate cancers, fallopian tube cancers, leiomyosarcomas, breast cancers, and ovarian cancers—diseases that are more canonically associated with copy number aberrations (Ciriello G et al. Nat Genet 2013, 45(10):1127-1133).

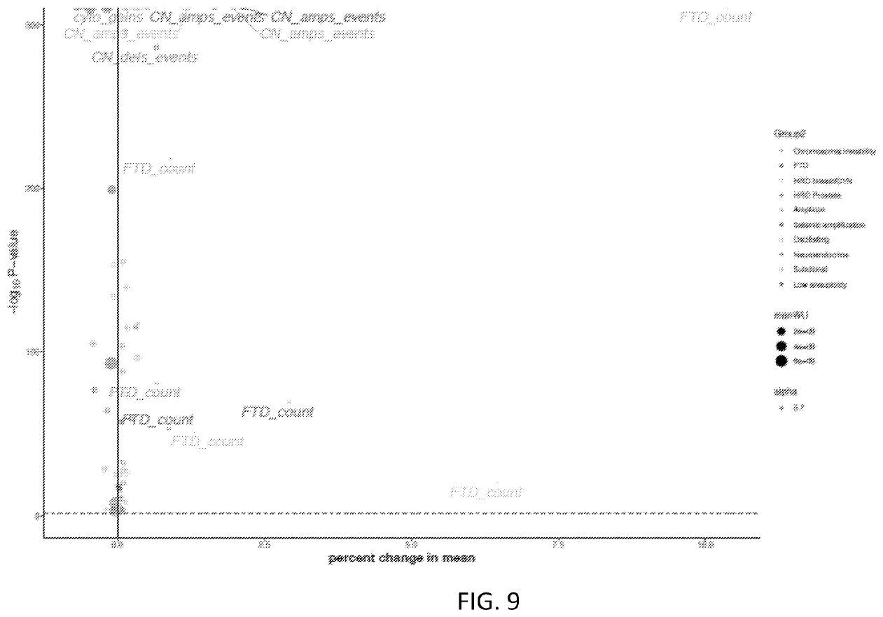

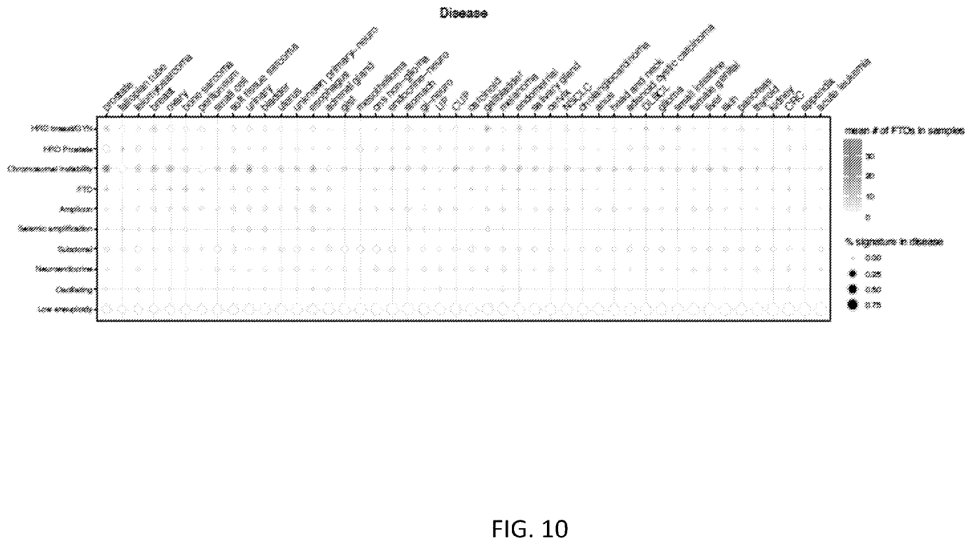

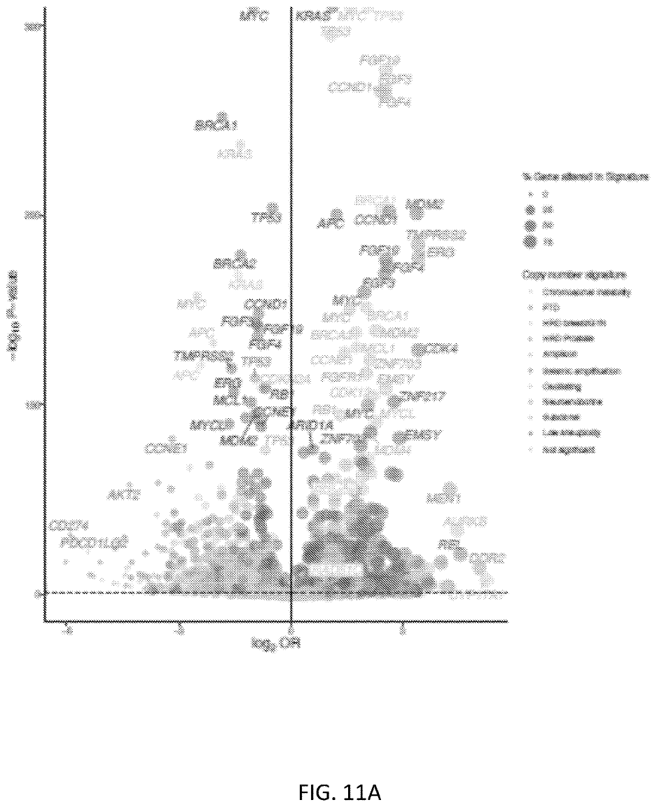

A chromosomal instability signature was characterized by moderate quantities of breakpoints (4 per chromosome arm) and shorter segments (<24 Mb) (FIGS. 7 & 8) and was most prevalent in fallopian tube cancers (25%), ovarian cancers (22%), and prostate cancers (19%). Chromosomal instability signature samples correlated with two mechanisms of chromosomal instability, focal tandem duplications (FTD) and gLOH (FIG. 6E). Samples harboring the chromosomal instability signature had 13% higher ploidies (mean of 3.1) than samples harboring other signatures (mean of 2.7) (FIG. 9).

A focal tandem duplication signature was identified and tumor samples in this signature averaged 24.5 FTD events vs 1.9 FTD events in samples outside of the signature (FIGS. 6E & 10). These FTD events are 10 kb and 10 Mb amplifications with single copy number gain (ploidy+1) (Sokol E S et al. Oncologist 2019, 24(12):1526-1533). The FTD signature was defined by having many breakpoints (10 per chromosome arm), very short segments (<13 Mb), segments at ploidy+1 and changepoints of 1 copy in diploid samples, which are all characteristic of an FTD phenotype.

A signature characteristic of HRD in breast/gynecologic (GYN) tumors was defined by both heterozygous deletions (segment copy numbers at half of ploidy) and duplications (ploidy+1 in diploid samples). Segment lengths represented wide range (7-51 Mb) and the profiles had few breakpoints (1 breakpoint per 25 Mb). This signature was highly enriched for high gLOH (>=16% genome under LOH; gLOH-high) samples (FIG. 6E) and HRD associated tumors including fallopian tube (13%), peritoneal (13%), ovarian (13%), and breast cancers (10%) (FIGS. 6A & 6C) (Sokol E S et al. JCO Precis Oncol 2020, 4:442-465). Another HRD signature was also identified and was characteristic of prostate tumors. The HRD prostate signature was defined by heterozygous deletions (copy number segments at half ploidy) but had segments at ploidy (segment copy number 1.6-2.2 for diploid samples), potentially driven by copy neutral loss of heterozygosity. Prostate tumors were highly enriched in this signature (21.7%; OR=12.4), whereas they were depleted in the HRD breast/GYN signature (OR=0.58) (FIG. 6C). While samples with the HRD prostate signature could be characterized by features associated with HRD, their relationship with gLOH was less clear. Using a threshold of 16% gLOH, they were weakly mutually exclusive (FIG. 6E). However, samples with the HRD prostate signature harbored a high median gLOH than samples negative for both HRD signatures (HRD prostate positive and HRD breast/GYN positive; [9.75% vs 7.72%, Mann Whitney U P value <4E-148]).

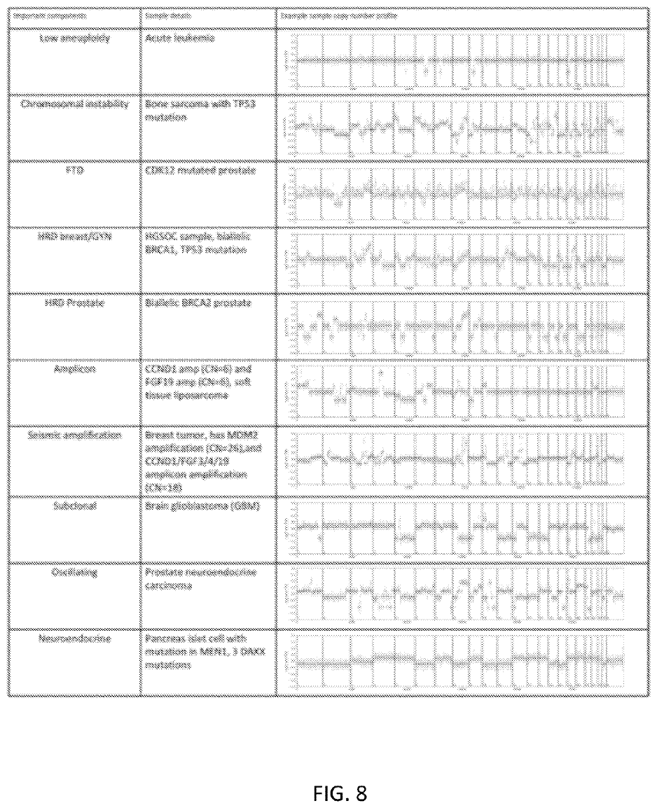

An amplicon signature and a seismic amplification signature were identified in this cohort. Both signatures were defined by the presence of high copy number amplifications, including amplifications at >70 copies gained (FIG. 6D). Samples with these signatures had an average of 4.6 and 4.8 known and likely pathogenic amplification alterations, amplicon and seismic amplification signatures respectively, compared to 1.3 of these amplifications outside of the signatures (FIG. 6D). The amplicon signature was defined by very short segments (2-7 Mb) amongst long segments (~72 Mb), whereas the seismic amplification showed seismic amplification patterns, containing segments of 7-13 Mb length and lacking long unbroken segments (FIG. 8). As seen by Rosswog and colleagues, breast, esophageal, and bladder cancers were highly prevalent in both amplification signatures, while the (non-seismic) amplicon signature was seen less frequently than the seismic amplification signature in uterine (1% vs 6%) and head and neck cancers (1% vs 4%) (FIG. 6C) (Rosswog C et al. Nat Genet 2021, 53(12):1673-1685).

A subclonal signature was characterized by clusters of breakpoints (5 per 50 Mb windows) leading to patterns of very short segments (1.7-3.7 Mb) and was most prevalent in gist (20%), CNS non-gliomas (18%), leiomyosarcoma (18%), small cell cancer (18%), and mesothelioma (15%). The subclonal signature was associated with an alkylating SBS signature (SBS11), called in 13% of subclonal signature samples (OR=9.9) (FIG. 6F). Moreover, this signature was defined by the presence of subploidy changepoints that centered around 0.27 copies for a diploid sample, reminiscent of copy number signature 5 from Macintyre and colleagues (Macintyre G et al. (2018) Nat Genet 50(9):1262-1270).

A neuroendocrine signature was defined by 7-13 Mb long segments with ploidy+1 copy number levels. The neuroendocrine signature positive samples averaged 10 cytogenetic gains vs 6.6 in samples outside of signature. The most prevalent diseases were adrenal gland cancers (11%), small cell cancers (11%), and hormone producing endocrine tumors (endocrine-neuro) cancers (10%).

An oscillating signature was characterized by having highly localized segmentation (3-5 breakpoints per 25 Mb) resulting in mix of shorter segments (1.7-7.1 Mb) and longer segments (42-72 Mb). The oscillating signature was also defined by long chains of oscillating copy number states (including-11 segment long chains). The localized chromosome shattering seen with this signature is a hallmark of chromothripsis (Stephens P J et al. Cell 2011, 144(1):27-40; Rausch T et al. Cell 2012, 148(1-2):59-71) and breakage fusion-bridges (Bignell G R et al. Genome Res 2007, 17(9):1296-1303).

Genomic Associations with Copy Number Signatures

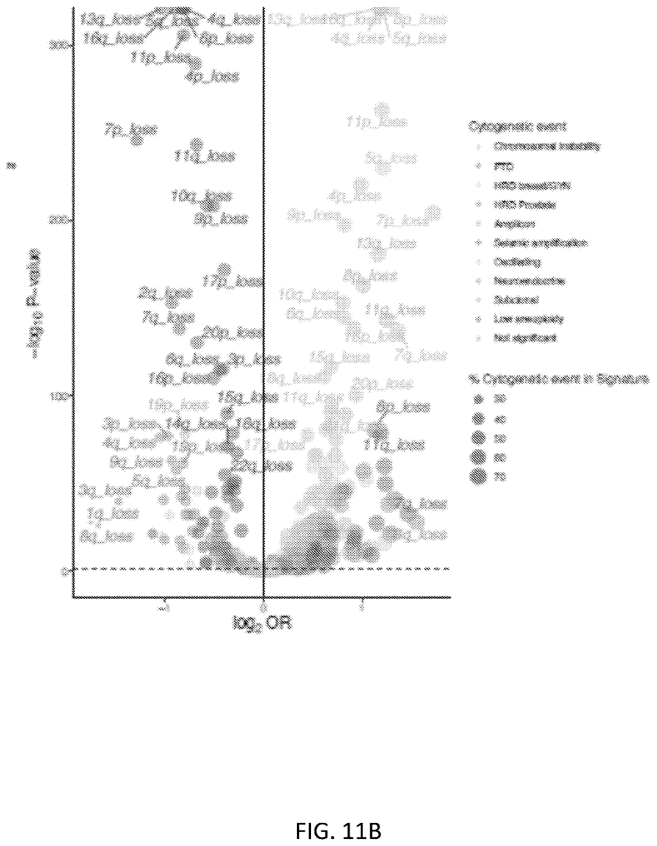

The association of copy number signatures with gene alterations, arm level cytogenetics events, and genomic features was examined (FIGS. 9, 11A, & 11B). Associations were found between these alterations and events with copy number signatures in a pan-cancer setting. Genes most often altered in samples with the low aneuploidy signature included KRAS, NRAS, APC, SMAD4, ARID1A, STK11 (FIG. 11A). The most commonly altered genes seen in the chromosomal instability signature were BRCA1, MCL1, BRCA2, and KDM5A. The chromosomal instability signature was also enriched for JUN (OR=3.3), SOX2 (OR=2.7), and JAK2 (OR=3.2) (FIG. 11A). These alterations were previously described as being associated with whole genome doubling in HGSOC (Macintyre G et al. (2018) Nat Genet 50(9):1262-1270). Samples with this signature were also associated with 18% higher telomeric content relative to samples without this signature (FIG. 9) and were positively associated with alterations in AURKB (OR=22), which may increase DNA damage through the loss of mediating the abscission checkpoint (Thoresen S B et al. Nat Cell Biol 2014, 16(6):550-560; Carlton J G et al. Science 2012, 336(6078):220-225). The FTD signature was enriched for CDK12 alterations (OR=6.6) which has been previously reported (FIG. 11A) (Sokol E S et al. Oncologist 2019, 24(12):1526-1533). Alterations in homologous recombination repair (HRR) pathway genes were associated with the HRD breast/GYN signature, including BRCA1(OR=5.5), BRCA2 (OR=2.3), and RAD51C (OR=2.5) (FIG. 11A). The HRD prostate signature was associated with BRCA2 (OR=3.9) and PALB2 (OR=1.9) alterations, reinforcing its association to HRD (FIG. 11A). Amplicon signature and seismic amplification signature were both enriched for the CCND1/FGF amplicon (FGF3/4/19) on chr11q (FIGS. 11A & 11B). The seismic amplification signature was enriched for mutations in genes associated with previous characterization of seismic amplifications, including: CDK4 (11%, OR=11), MDM2 (12%, OR=11), CCND1 (8%, OR=6), FGFR1 (12%, OR=4), ERBB2 (13%, OR=4), and AKT2 (4%, OR=3) (FIG. 11A) (Rosswog C et al. Nat Genet 2021, 53(12):1673-1685). The subclonal signature was enriched for alterations in IDH1 (OR=3), CDKN2C (OR=3), and RB1 (OR=2), and for 13q arm loss (FIGS. 11A & 11B). The neuroendocrine signature was highly enriched for MEN1(OR=21.4). MEN1, Multiple Neoplasia type 1, is known to predispose patients for multiple endocrine tumors (Bergman L et al. Br J Cancer 2000, 83(8):1003-1008). The oscillating copy number signature was enriched for alterations in MAP2K4 (OR=3), DNMT3A (OR=2), and ESR1 (OR=2) (FIG. 11A).

### HRD Copy Number Signatures Capture Genomic BRCAness Scarring

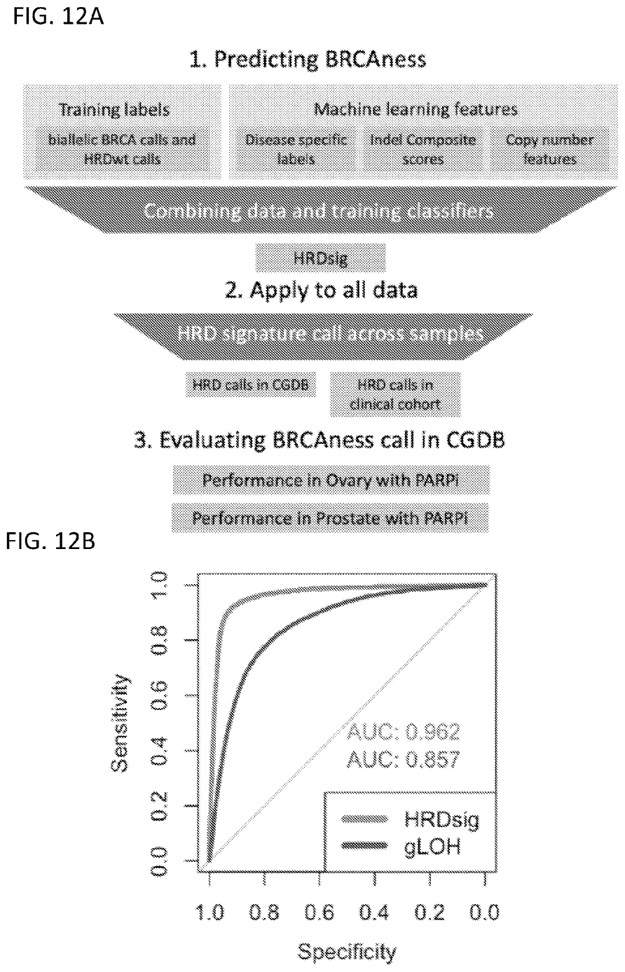

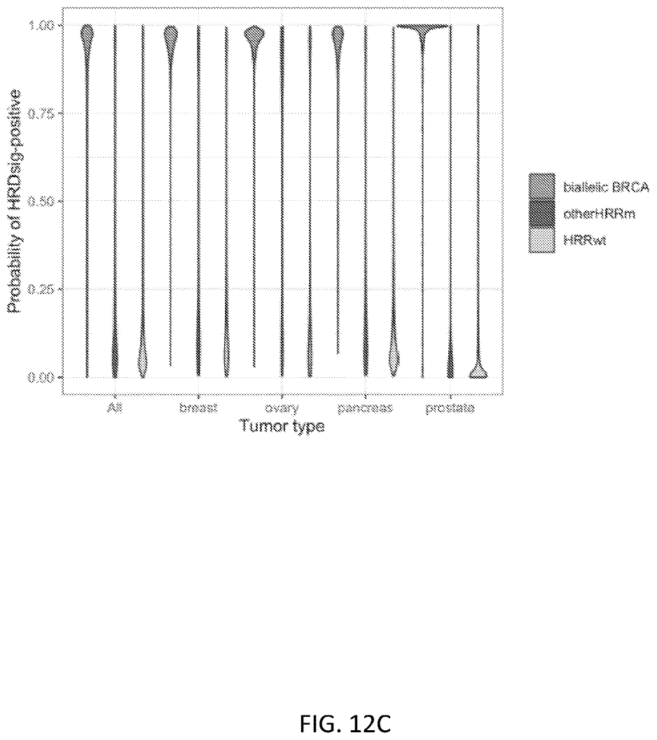

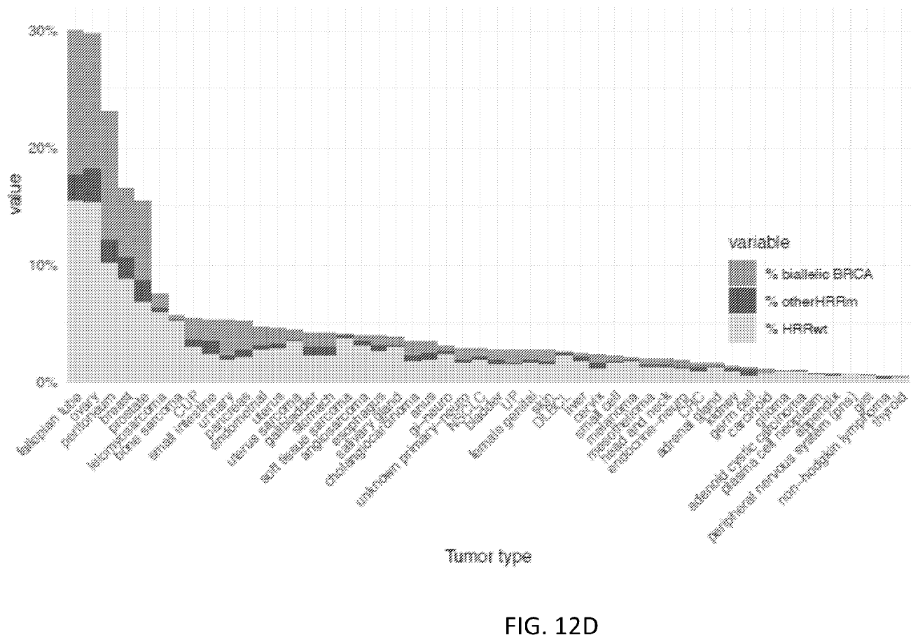

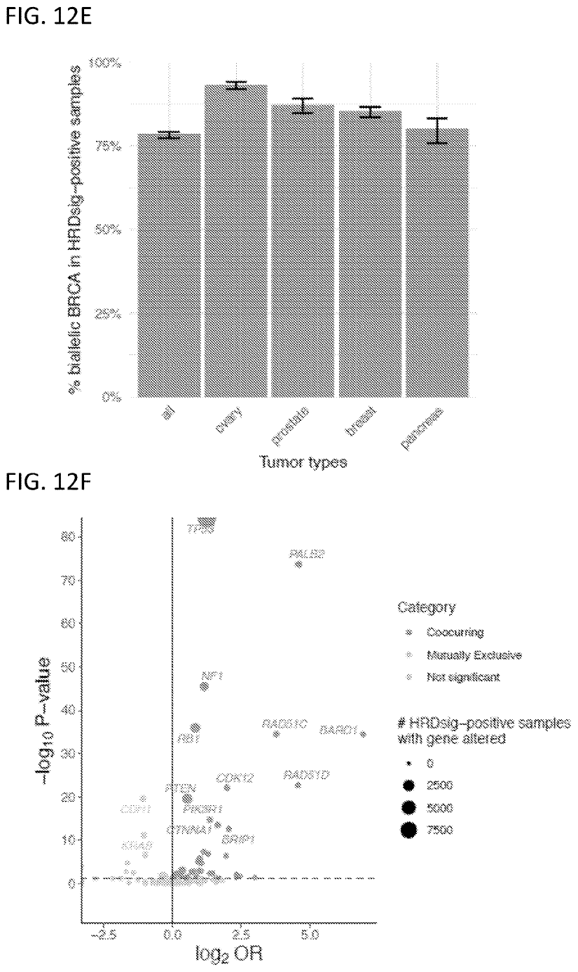

The generation of copy number signatures revealed multiple associated with HRD (HRD breast/GYN signature and HRD prostate signature), suggesting that copy number features are informative in detecting relevant genomic scars. Previous work had identified an association of indels within microhomology regions and HRD (Alexandrov L B et al. Nature 2020, 578(7793):94-101). Since copy number and indel features may contain orthogonal information and may better identify BRCAness, we utilized both sets of features to develop an extreme gradient boosting (XGB) machine learning classifier to detect HRD positivity (HRDsig) (FIG. 12A). Using biallelic BRCA1/2 as true positives and homologous recombination repair wildtype (HRRwt) samples as true negatives, the HRDsig algorithm outperformed gLOH overall and across diseases (FIG. 12B). Importantly, the algorithm identified a population of HRDsig-positive samples even within the population lacking BRCA1/2 or other HRR alterations, especially in breast and ovarian cancers where non-genomic (e.g. methylation) mechanisms of HRD have been reported (FIGS. 12C & 12D) (Ruscito I et al. Eur J Cancer 2014, 50(12):2090-2098; Yamashita N et al. Clin Breast Cancer 2015, 15(6):498-504). Sensitivity to detect biallelic BRCA1/2 alterations was high across HRD-associated tumors; ovarian cancers (93%), prostate cancers (87%), breast cancers (85%), pancreatic cancers (80%) (FIG. 12E). Across the pan-cancer dataset, the prevalence of HRDsig positivity was 6.4% with the highest frequencies in fallopian tube cancers (30%), ovarian cancers (30%), peritoneal cancers (23%), breast cancers (16%), and prostate cancers (15%) (FIG. 12D). Diseases with the lowest prevalence of HRDsig included thyroid cancers (0.5%), acute leukemias (0.2%), and myelodysplastic syndromes (0.1%). In the BRCA1/2 wildtype (BRCAwt) population, HRDsig positivity was strongly associated with biallelic alterations in BARD1(OR=122, p<1E-10), PALB2 (OR=24, p<1E-10), RAD51D (OR=24, p<1E-10), and RAD51C (OR=14, p<1E-10) (FIG. 12F).

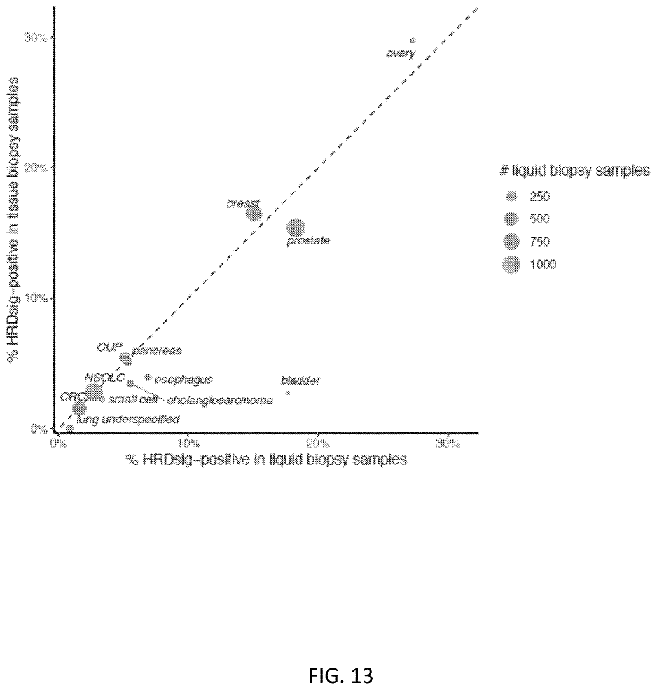

While the algorithm was trained using tissue biopsy samples, liquid biopsies are able to detect indels and copy number events when sufficient circulating tumor DNA (ctDNA) is present (Woodhouse R et al. PLoS One 2020, 15(9):e0237802). Therefore, the frequency of HRDsig was examined in high shedding samples (tumor fraction >10%) using an unmodified algorithm. The frequency of HRDsig-positivity was similar in tissue and liquid across diseases (e.g. 30% v 27% for tissue and liquid in ovarian cancer, 15% v 18% in prostate, and 16% v 15% in breast cancer) (FIG. 13). These findings suggest the HRD signatures may be applicable to liquid biopsy samples when sufficient ctDNA is present, though additional optimization may be required.

### HRDsig Predicts PARPi Benefit in Ovarian and Prostate Clinical Cohorts

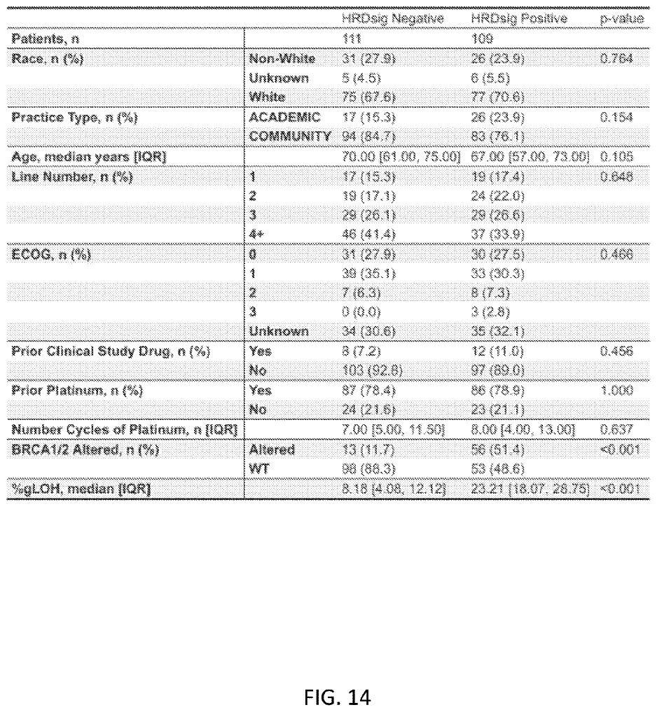

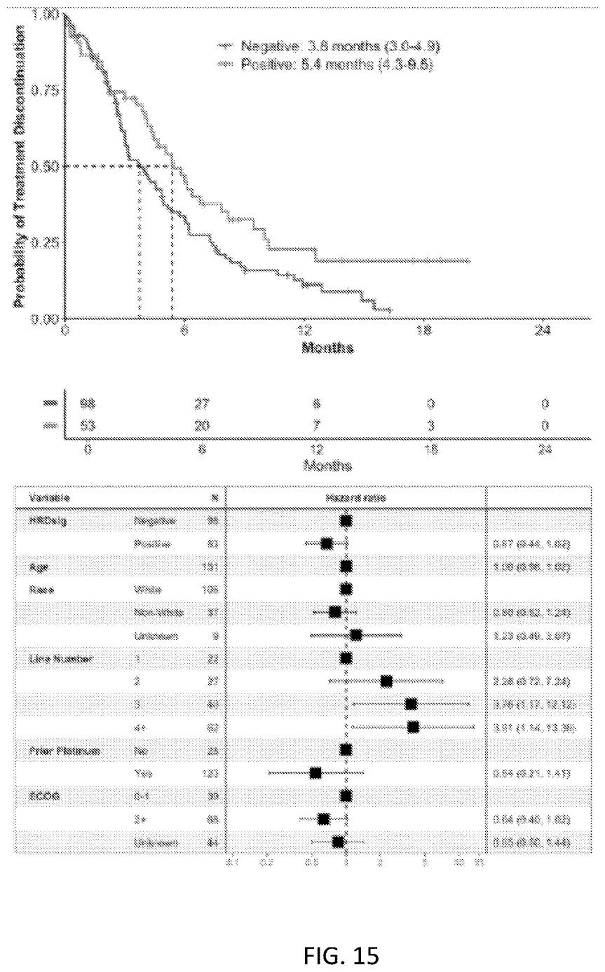

A real-world clinico-genomic database (CGDB) was leveraged to examine the association of HRDsig positivity with outcomes on PARPi for patients with ovarian cancer (FIG. 5A). 220 patients were treated with PARPi following CGP specimen collection (HRDsig-positive=109; negative=111). HRDsig-positive patients were clinically similar to HRDsig-negative patients (FIG. 14) but had higher rates of BRCA1/2 mutations and higher gLOH. HRDsig positivity was associated with improved time to treatment discontinuation (TTD; median 9.0 mo v 4.0 mo; multivariate HR=0.50 [0.36-0.70], p<0.001). When limited to BRCAwt ovarian cancer patients treated with PARPi, HRDsig continued to select patients with numerically longer TTD (median 5.4 mo v 3.8 mo; multivariate HR=0.67 [0.44-1.02], p=0.06) (FIG. 15).

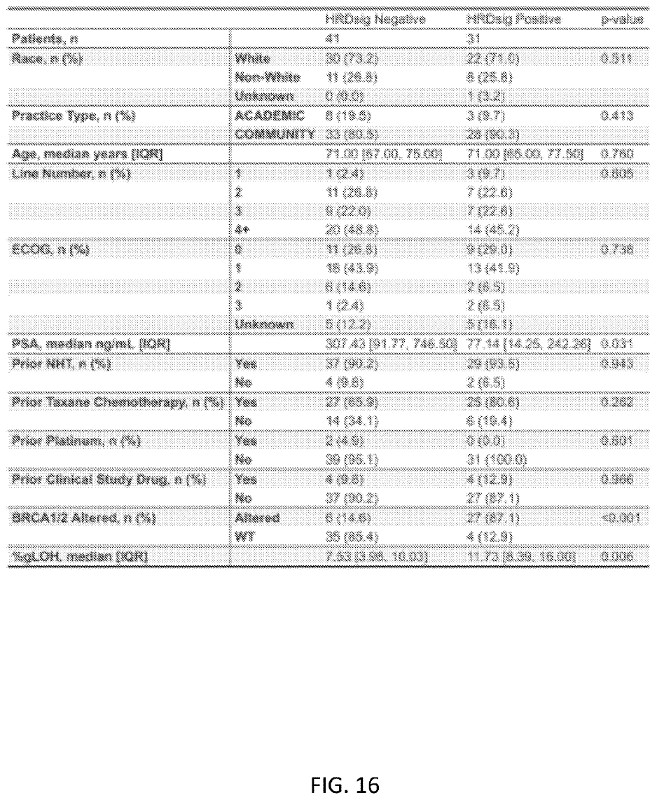

In a prostate CGDB 72 metastatic castration-resistant prostate cancer (mCRPC) patients (FIG. 5D) were treated with PARPi (HRDsig-positive=31; negative=41). HRDsig-positive patients were clinically similar to HRDsig-negative patients (FIG. 16) with the exception of BRCA1/2 mutation rate and associations with gLOH. HRDsig was significantly associated with prolonged TTD on PARPi (median 6.8 mo v 3.2 mo; HR=0.50 [0.26-0.96], p=0.036).

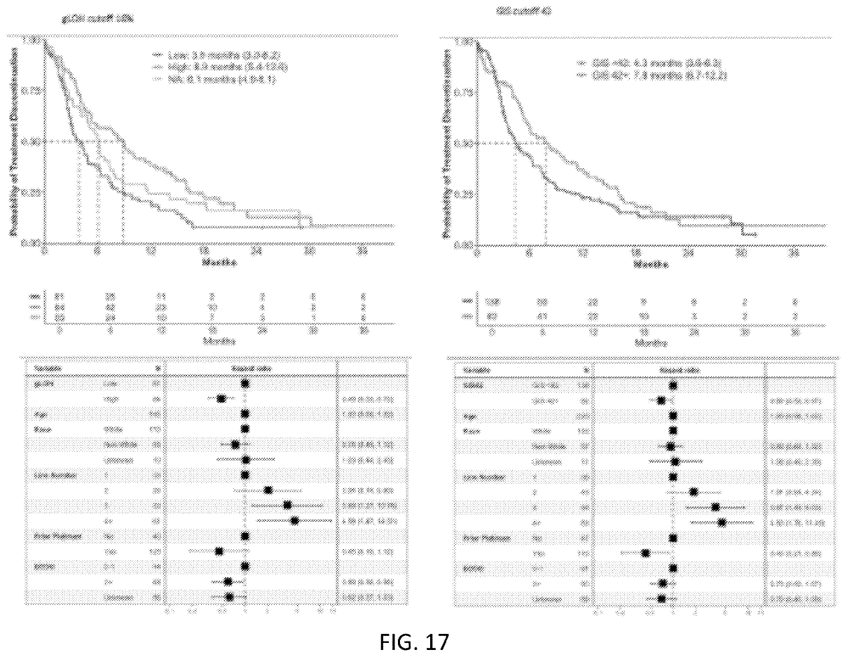

Genome-wide LOH and genome instability are currently utilized in ovarian cancer for identifying patients who may benefit from PARPi. The performance of these measures was examined in stratifying PARPi TTD in ovarian cancer (FIG. 17). gLOH (≥16%) and GIS (≥42) were associated with significant PARPi TTD (gLOH HR 0.49 [0.33-0.72]p<0.001; GIS HR 0.69 [0.50-0.97]p=0.03).

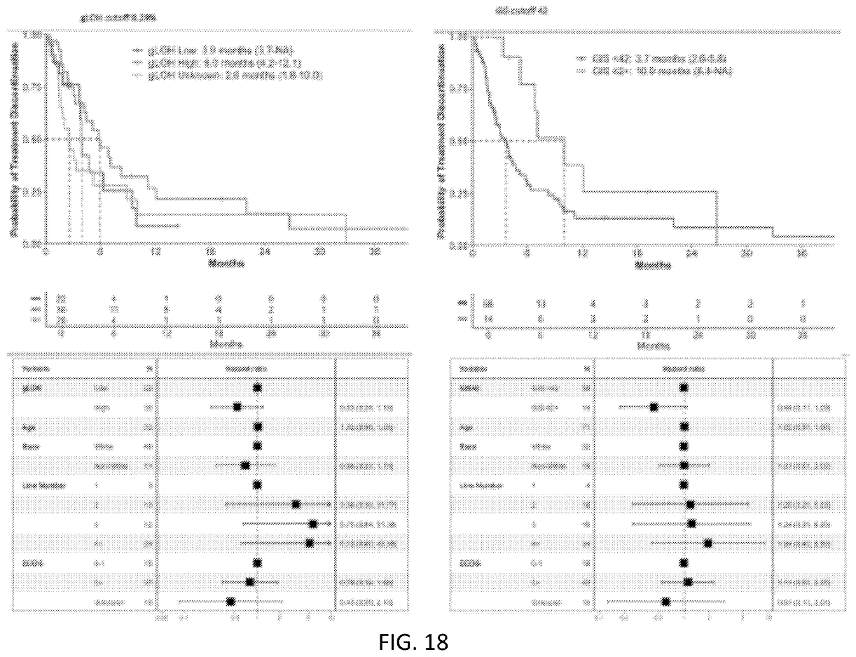

gLOH (>8.29%) and GIS (≥42) were also examined in prostate cancer. While both gLOH and GIS identified fewer patients than HRDsig (gLOH: 22 v 31; GIS: 14 v 31), each trended predictive for PARPi TTD (gLOH-high: HR=0.53 [0.24-1.18]p=0.3; GIS>42: HR=0.44 [0.17-1.09], p=0.08) (FIG. 18).

### Discussion

This study expands on prior work exploring copy number signatures in HGSOC (Macintyre G et al. (2018) Nat Genet 50(9):1262-1270), prostate (Wang S et al. PLoS Genet 2021, 17(5):e1009557), multiple breast (Pladsen A V et al. Commun Biol 2020, 3(1):153), myeloma (Maclachlan K H et al. Nat Commun 2021, 12(1):5172), urothelial carcinoma (Guan B et al. Front Cell Dev Biol 2021, 9:713499), and pan-cancer (Steele C D et al. Signatures of copy number alterations in human cancer. bioArxiv 2021) settings by investigating over 259,000 samples pan-cancer. These results build upon Steele et al. by applying copy number signature calling to genomic profiles obtained from targeted panel sequencing assays—which currently have widespread clinical usage. Additional copy number signatures were discovered with strong disease bias and genomic associations. It was found that 29% of tumors had dominant copy number signature associated with chromosomal instability, with the highest rates observed in prostate cancers (56%), fallopian tube cancers (53%), and leiomyosarcomas (52%), whereas tumors harboring a dominant copy number signature associated with quiet copy number genomes were most often acute leukemias (90%), appendiceal cancers (88%) and colorectal cancers (87%), where copy number variant processes were less common. The mutual exclusivity of complex copy number profiles and driver alterations has been reported previously (Ciriello G et al. Nat Genet 2013, 45(10):1127-1133), and underscores the need to better understand the biological and clinical implications of these signatures. In the pan-cancer cohort, 30% of samples had an aberrant copy number signature. In particular, 5% of all samples were associated with one of the two HRD signatures representing a population that could benefit from PARPi.

PARP inhibitors improve outcomes in HR deficient patients in a number of tumor types. Current patient selection strategies primarily rely on the presence of germline and/or tumor alterations in BRCA1 and BRCA2. These strategies have been successful in ovarian, prostate, pancreatic, and breast cancer; however, beyond these BRCA-associated cancer types, such a biomarker strategy may have pitfalls. BRCA1/2 alterations, even those of germline origin, may not contribute to the pathogenicity of diseases such as lung cancer or colorectal cancer. Furthermore, in diseases with high mutational burden, these BRCA1/2 alterations may represent monoallelic passenger alterations. Sokol et al. reported that while a majority of BRCA1/2 alterations are biallelic in ovary (95%), breast (88%), prostate (87%), and pancreatic (79%) cancers, in most other tumor types, a minority of alterations are predicted biallelic, including low rates in melanoma (17%) and skin squamous cell carcinoma (33%) (Sokol E S et al. JCO Precis Oncol 2020, 4:442-465). These monoallelic alterations are not associated with elevated genomic scarring and with the remaining functional copy, likely do not result in functional HRD. Consistent with this, the LUNG-MAP trial recently reported low response rates in a PARPi treated biallelic-BRCA NSCLC cohort (Owonikoko T K et al. (2021) Clin Lung Cancer 22(3):187-194.el). Thus, a mutation-only approach poses a diagnostic challenge for PARPi selection.

Potential alternative approaches involve assessing HRD through RAD51 foci formation or through quantification of copy number scarring. In the ARIEL3 trial, focal genome-wide LOH was associated with longer progression free survival in both BRCA1/2-mutated and BRCA1/2-wildtype ovarian cancer (Coleman R L et al. Lancet 2017, 390(10106):1949-1961). Similarly, in PAOLA-1, a copy-number based genomic instability score was associated with benefit of combination olaparib and bevacizumab in ovarian cancer (Ray-Coquard I et al. N Engl J Med 2019, 381(25):2416-2428). While successful in ovarian cancer, it's not clear if these biomarkers will be more broadly applicable across tumor types. In particular, gLOH as well as GIS scores exhibit relatively poor ability to separate biallelic BRCA1/2 mutant and BRCA1/2 wildtype samples in prostate cancer (Sokol E S et al. JCO Precis Oncol 2020, 4:442-465; Lotan T L et al. Mod Pathol 2021, 34(6):1185-1193). A more nuanced biomarker may be required for robust prediction of HRD across tumor types. Recently, Leibowitz et al. released a pre-print article describing a HRD algorithm in NSCLC, ovarian, prostate, pancreatic, and breast cancer (Leibowitz B et al. medRxiv 2021, 2021.12.20.21267985). While no clinical data was presented, high F1-scores were observed. Unfortunately, the model was developed with an extremely limited set of data (75% of samples were labeled as ambiguous), awaiting validation on a broad, representative population. These findings suggest that HRD is associated with genomic scarring beyond ovarian cancer.

This example describes an HRD signature trained with a diverse set of copy number and indel features. While the highest rates of positivity were seen in fallopian tube, ovarian, peritoneal, breast, and prostate cancer, appreciable frequencies were seen across tumor types, with a 6% overall prevalence. HRDsig was associated with biallelic BRCA1/2 alterations was high across tumor types. Importantly, HRDsig identified a BRCA1/2 wildtype population enriched for biallelic alterations in other HRR genes and in some cases lacking any HRR alteration, suggesting that HRDsig is able to detect a BRCAness population not currently eligible for PARPi in most indications. In ovarian and prostate cancer clinical cohorts, HRDsig was predictive of improved outcomes on PARPi efficacy, with similar or trending superior performance relative to gLOH and GIS. In ovarian cancer, sample numbers were sufficient to demonstrate predictive power of HRDsig in the BRCAwt population. These findings suggest that HRDsig has potential utility in selecting a broader population of patients that may benefit from PARPi use. The results in prostate cancer suggest that HRD signatures may have utility in predicting PARPi response beyond ovarian cancer.

Characterization of pan-cancer copy number signatures revealed distinct molecular subtypes across a wide array of cancers. Many of these signatures were present in almost all tumor types, reflecting an opportunity for the use of pattern-based scores in targeted therapy selection in pan-cancer trials. Even for HRD, which represents a well appreciated and targeted phenotype, it was found that patterns of scarring differed across tumor types with prostate-specific patterns, which requires a nuanced genomic scar-based signature. The algorithm was developed using a hybrid capture targeted panel and may be applicable to other panel-based tests including liquid biopsies, which would be valuable in cases where limited tissue is available, or a post-progression sample is preferred.

In summary, the landscape of 10 copy number signatures was described in a pan-cancer cohort of 259,415 tumor samples, identifying associations with genomic alterations and clinical characteristics. Copy number signatures were seen at a high prevalence across many cancer types, and all 10 signatures were present in 41/46 of cancer types analyzed, highlighting their broad relevance. The results demonstrated that data underlying copy number signatures and copy number features can be utilized for a machine learning based homologous recombination deficiency caller that had predictive value for PARP inhibitor benefit in real-world datasets for ovarian and prostate cancer. This highlighted that characterizing copy number signatures on a 300 gene hybrid capture targeted panel assay in many cancer types may have use as a clinical decision-making tool and extend current understanding of the biology of copy number aberrations in cancer.

### Exemplary Implementations

Exemplary implementations of the methods and systems described herein include:

1. A method comprising:
providing a plurality of nucleic acid molecules obtained from a sample from an individual diagnosed with or suspected of having a disease;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, targeted sequence read data for the sample derived from the individual;
determining, using the one or more processors, a copy number profile having a plurality of copy number features for the individual based on the targeted sequence read data for the sample;
determining, using the one or more processors, a copy number feature value for each of the plurality of copy number features;
performing, using the one or more processors, an analysis of the copy number feature values for each of a plurality of copy number features using a first trained model to determine a probability that each of one or more copy number feature subgroups is represented in the copy number profile for the sample;
performing, using the one or more processors, an analysis of the probabilities determined for each of the one or more copy number feature subgroups using a second trained model to identify one or more copy number signatures represented in the copy number profile for the individual, and to determine one or more corresponding copy number signature scores;
comparing, using the one or more processors, the copy number signature scores for the one or more identified copy number signatures to one or more corresponding predetermined thresholds; and
identifying, based on the comparison, a dominant copy number signature for the individual; or
identifying, based on the comparison, one or more copy number signatures that are components of the copy number profile for the individual.
   2. The method of clause 1, further comprising confirming a diagnosis of the disease for the individual based on the dominant copy number signature or the one or more copy number signatures identified for the individual.
   3. The method of clause 1, further comprising predicting a treatment outcome for the individual based on the dominant copy number signature or the one or more copy number signatures identified for the individual.
   4. The method of clause 1, further comprising selecting a treatment for the individual based on the dominant copy number signature or the one or more copy number signatures identified for the individual.
   5. The method of clause 1, further comprising adjusting a treatment dosage for the individual based on the dominant copy number signature or the one or more copy number signatures identified for the individual.
   6. The method of clause 1, further comprising selecting the individual for inclusion in a clinical trial based on the dominant copy number signature or the one or more copy number signatures identified for the individual.
   7. The method of clause 1, further comprising identifying the individual as a candidate for chemotherapy, radiation therapy, or surgery based on the dominant copy number signature or the one or more copy number signatures identified for the individual.
   8. The method of clause 1, further comprising identifying the individual as a candidate for hospice care based on the dominant copy number signature or the one or more copy number signatures identified for the individual.
   9. The method of any one of clauses 1 to 8, wherein the copy number profile for the individual is determined based on single nucleotide polymorphisms (SNPs) located in the targeted sequence read data for the sample.
   10. The method of clause 9, wherein the single nucleotide polymorphisms (SNPs) are located in on-target sequence read data.
   11. The method of any one of clauses 1 to 10, wherein the copy number profile for the individual is determined based on off-target sequence reads in the targeted sequence read data for the sample.
   12. The method of any one of clauses 1 to 11, wherein the copy number profile for the individual is determined based on a combination of on-target and off-target sequence reads in the targeted sequence read data for the sample.
   13. The method of any one of clauses 1 to 12, wherein the analysis to identify one or more copy number signatures represented in the copy number profile of the individual comprises an analysis of both the probabilities determined for each of the one or more copy number feature subgroups and a presence of one or more genomic features detected in the targeted sequence read data for the individual using the second trained model.
   14. The method of clause 13, wherein the one or more genomic features comprise one or more short variants, one or more indels, one or more biallelic loss-of-function variants, one or more rearrangements, or any combination thereof.
   15. The method of any one of clauses 1 to 14, wherein the subject is suspected of having or is determined to have cancer.
   16. The method of clause 15, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
   17. The method of clause 15, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
   18. The method of any one of clauses 15 to 17, further comprising treating the subject with an anti-cancer therapy.
   19. The method of clause 18, wherein the anti-cancer therapy comprises a targeted anti-cancer therapy.
   20. The method of clause 19, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
   21. The method of any one of clauses 1 to 20, further comprising obtaining the sample from the subject.
   22. The method of any one of clauses 1 to 21, wherein the sample comprises a tissue biopsy sample, a liquid biopsy sample, or a normal control.
   23. The method of clause 22, wherein the sample is a liquid biopsy sample and comprises blood, plasma, cerebrospinal fluid, sputum, stool, urine, or saliva.
   24. The method of clause 22, wherein the sample is a liquid biopsy sample and comprises circulating tumor cells (CTCs).
   25. The method of clause 22, wherein the sample is a liquid biopsy sample and comprises cell-free DNA (cfDNA), circulating tumor DNA (ctDNA), or any combination thereof.
   26. The method of any one of clauses 1 to 25, wherein the plurality of nucleic acid molecules comprises a mixture of tumor nucleic acid molecules and non-tumor nucleic acid molecules.
   27. The method of clause 26, wherein the tumor nucleic acid molecules are derived from a tumor portion of a heterogeneous tissue biopsy sample, and the non-tumor nucleic acid molecules are derived from a normal portion of the heterogeneous tissue biopsy sample.
   28. The method of clause 27, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
   29. The method of any one of clauses 1 to 28, wherein the one or more adapters comprise amplification primers, flow cell adaptor sequences, substrate adapter sequences, or sample index sequences.
   30. The method of any one of clauses 1 to 29, wherein the captured nucleic acid molecules are captured from the amplified nucleic acid molecules by hybridization to one or more bait molecules.
   31. The method of clause 30, wherein the one or more bait molecules comprise one or more nucleic acid molecules, each comprising a region that is complementary to a region of a captured nucleic acid molecule.
   32. The method of any one of clauses 1 to 31, wherein amplifying nucleic acid molecules comprises performing a polymerase chain reaction (PCR) amplification technique, a non-PCR amplification technique, or an isothermal amplification technique.
   33. The method of any one of clauses 1 to 32, wherein the sequencing comprises use of a massively parallel sequencing (MPS) technique, whole genome sequencing (WGS), whole exome sequencing, targeted sequencing, direct sequencing, or Sanger sequencing technique.
   34. The method of clause 33, wherein the sequencing comprises massively parallel sequencing, and the massively parallel sequencing technique comprises next generation sequencing (NGS).
   35. The method of any one of clauses 1 to 34, wherein the sequencer comprises a next generation sequencer.
   36. The method of any one of clauses 1 to 35, wherein one or more of the plurality of sequencing reads overlap one or more gene loci within one or more subgenomic intervals in the sample.
   37. The method of clause 36, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
   38. The method of clause 36 or clause 37, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (Cllorf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
   39. The method of clause 36 or clause 37, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-10, IL-6, IL-6R, JAK1, JAK2, JAK3, KIT, KRAS, MEK, MET, MSI-H, mTOR, PARP, PD-1, PDGFR, PDGFRa, PDGFRP, PD-L1, PI3K6, PIGF, PTCH, RAF, RANKL, RET, ROS1, SLAMF7, VEGF, VEGFA, VEGFB, or any combination thereof.
   40. The method of any one of clauses 1 to 39, further comprising generating, by the one or more processors, a report indicating the dominant copy number signature or the one or more copy number signature components identified for the individual.
   41. The method of clause 40, further comprising transmitting the report to a healthcare provider.
   42. The method of clause 41, wherein the report is transmitted via a computer network or a peer-to-peer connection.
   43. A method, comprising:
receiving, at one or more processors, targeted sequence read data for a sample derived from an individual diagnosed with or suspected of having a disease;
determining, using the one or more processors, a copy number profile having a plurality of copy number features for the individual based on the targeted sequence read data for the sample;
determining, using the one or more processors, a copy number feature value for each of the plurality of copy number features;
performing, using the one or more processors, an analysis of the copy number feature values for each of a plurality of copy number features using a first trained model to determine a probability that each of one or more copy number feature subgroups is represented in the copy number profile for the sample;
performing, using the one or more processors, an analysis of the probabilities determined for each of the one or more copy number feature subgroups using a second trained model to identify one or more copy number signatures represented in the copy number profile for the individual, and to determine one or more corresponding copy number signature scores;
comparing, using the one or more processors, the copy number signature scores for the one or more identified copy number signatures to one or more corresponding predetermined thresholds; and
identifying, based on the comparison, a dominant copy number signature for the individual; or
identifying, based on the comparison, one or more copy number signatures that are components of the copy number profile for the individual.
   44. The method of clause 43, further comprising confirming a diagnosis of the disease for the individual based on the dominant copy number signature or the one or more copy number signatures identified for the individual.
   45. The method of clause 43, further comprising predicting a treatment outcome for the individual based on the dominant copy number signature or the one or more copy number signatures identified for the individual.
   46. The method of clause 43, further comprising selecting a treatment for the individual based on the dominant copy number signature or the one or more copy number signatures identified for the individual.
   47. The method of clause 43, further comprising adjusting a treatment dosage for the individual based on the dominant copy number signature or the one or more copy number signatures identified for the individual.
   48. The method of clause 43, further comprising selecting the individual for inclusion in a clinical trial based on the dominant copy number signature or the one or more copy number signatures identified for the individual.
   49. The method of clause 43, further comprising identifying the individual as a candidate for chemotherapy, radiation therapy, or surgery based on the dominant copy number signature or the one or more copy number signatures identified for the individual.
   50. The method of clause 43, further comprising identifying the individual as a candidate for hospice care based on the dominant copy number signature or the one or more copy number signatures identified for the individual.
   51. The method of any one of clauses 43 to 50, wherein the copy number profile for the individual is determined based on single nucleotide polymorphisms (SNPs) located in the targeted sequence read data for the sample.
   52. The method of clause 51, wherein the single nucleotide polymorphisms (SNPs) are located in on-target sequence read data.
   53. The method of any one of clauses 43 to 52, wherein the copy number profile for the individual is determined based on off-target sequence reads in the targeted sequence read data for the sample.
   54. The method of any one of clauses 43 to 53, wherein the copy number profile for the individual is determined based on a combination of on-target and off-target sequence reads in the targeted sequence read data for the sample.
   55. The method of clause 53 or clause 54, wherein the off-target sequence reads comprise an average sequencing coverage of less than 1.0×, 0.9×, 0.8×, 0.7×, 0.6×, 0.5×, 0.4×, 0.3×, 0.2×, or 0.1×.
   56. The method of any one of clauses 43 to 55, wherein the analysis to identify one or more copy number signatures represented in the copy number profile of the individual comprises an analysis of both the probabilities determined for each of the one or more copy number feature subgroups and a presence of one or more genomic features detected in the targeted sequence read data for the individual using the second trained model.
   57. The method of clause 56, wherein the one or more genomic features comprise one or more short variants, one or more indels, one or more biallelic loss-of-function variants, one or more rearrangements, or any combination thereof.
   58. The method of any one of clauses 43 to 57, wherein the plurality of copy number features comprises a number of breakpoints per 10 Mb of genomic sequence, a number of breakpoints per 25 Mb of genomic sequence, a number of breakpoints per 50 Mb of genomic sequence, a number of breakpoints per 100 Mb of genomic sequence, a number of breakpoints per chromosome arm for each chromosome, a magnitude of a copy number change between any two adjacent copy number segments; a length of each copy number segment; the copy number of segments; a count of contiguous oscillating copy number chains, or any combination thereof.
   59. The method of any one of clauses 43 to 58, wherein the first trained model comprises a trained statistical model.
   60. The method of any one of clauses 43 to 58, wherein the first trained model comprises a trained machine learning model.
   61. The method of any one of clauses 43 to 60, wherein the first trained model is updated based on the one or more copy number signatures identified in the copy number profile of the individual.
   62. The method of any one of clauses 43 to 61, wherein the first trained model comprises a latent class analysis model or a mixture model.
   63. The method of any one of clauses 43 to 62, wherein the first trained model is trained on copy number feature values calculated for each of the plurality of copy number features based on copy number profiles for a cohort of individuals diagnosed with the disease.
   64. The method of clause 63, wherein the disease comprises a cancer.
   65. The method of clause 64, wherein the cancer comprises ovarian cancer, prostate cancer, or colorectal cancer.
   66. The method of any one of clauses 43 to 65, wherein the second trained model comprises a non-negative matrix factorization (NMF) model or a machine learning model.
   67. The method of clause 66, wherein the second trained model comprises a machine learning model, and the machine learning model comprises a supervised, semi-supervised, or unsupervised machine learning model.
   68. The method of clause 67, wherein the second trained model comprises a supervised machine learning model, and the supervised machine learning model comprises an XGBoost model.
   69. The method of any one of clauses 43 to 68, wherein the second trained model is trained on copy number feature subgroups identified in data for a cohort of individuals diagnosed with the disease.
   70. The method of any one of clauses 43 to 69, wherein the second trained model is trained on copy number feature subgroups identified in data for a cohort of individuals comprising individuals diagnosed with a plurality of different diseases.
   71. The method of clause 70, wherein the plurality of different diseases comprises a plurality of different cancers.
   72. The method of clause 71, wherein the plurality of different cancers comprises ovarian cancer, breast cancer, non-small cell lung carcinoma (NSCLC), esophageal cancer, prostate cancer, pancreatic cancer, colorectal cancer, or any combination thereof.
   73. The method of any one of clauses 43 to 72, wherein the second trained model is trained to identify copy number signatures based on the probabilities determined for each of the one or more copy number feature subgroups and based on the presence of one or more additional genomic features identified in the targeted sequence read data.
   74. The method of clause 73, wherein the one or more additional genomic features comprise one or more short variants, one or more indels, one or more biallelic loss-of-function variants, one or more rearrangements, or any combination thereof.
   75. The method of any one of clauses 43 to 74, wherein the one or more corresponding predetermined thresholds are determined based on an analysis of copy number signature scores for a cohort of individuals diagnosed with the disease.
   76. The method of any one of clauses 43 to 75, wherein the one or more corresponding predetermined thresholds are determined based on an analysis of copy number signature scores for a cohort of individuals comprising individuals diagnosed with a plurality of different diseases.
   77. The method of any one of clauses 43 to 76, wherein the one or more corresponding predetermined thresholds are determined based on an analysis of data for a cohort of individuals that comprises both copy number signature scores and clinical data.
   78. The method of clause 77, wherein the clinical data comprises the individual's sex, age, gender, height, weight, clinical history, family history, sample type, tumor stage, tumor grade, or any combination thereof.
   79. The method of any one of clauses 43 to 78, wherein the identification of a dominant copy number signature or of one or more copy number signature components is used to diagnose or confirm a diagnosis of disease in the individual.
   80. The method of clause 79, wherein the disease is cancer.
   81. The method of clause 80, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
   82. The method of clause 80 or clause 81, further comprising selecting an anti-cancer therapy to administer to the subject based on the identification of the dominant copy number signature or of the one or more copy number signature components.
   83. The method of any one of clauses 80 to 82, further comprising determining an effective amount of an anti-cancer therapy to administer to the subject based on the identification of the dominant copy number signature or of the one or more copy number signature components.
   84. The method of clause 82 or clause 83, further comprising administering the anti-cancer therapy to the subject based on the identification of the dominant copy number signature or of the one or more copy number signature components.
   85. The method of any one of clauses 82 to 84, wherein the anti-cancer therapy comprises chemotherapy, radiation therapy, immunotherapy, a targeted therapy, or surgery.
   86. A method for diagnosing a disease, the method comprising:
diagnosing that a subject has the disease based on an identification of a dominant copy number signature or of one or more copy number signature components in a copy number profile for a sample from an individual, wherein the dominant copy number signature or the one or more copy number signature components are identified according to the method of any one of clauses 43 to 78.
   87. A method of selecting an anti-cancer therapy, the method comprising:
responsive to an identification of a dominant copy number signature or of one or more copy number signature components in a copy number profile for a sample from an individual, selecting an anti-cancer therapy for the individual, wherein the dominant copy number signature or the one or more copy number signature components are identified according to the method of any one of clauses 43 to 78.
   88. A method of treating a cancer in an individual, comprising:
responsive to an identification of a dominant copy number signature or of one or more copy number signature components in a copy number profile for a sample from an individual, administering an effective amount of an anti-cancer therapy to the individual, wherein the dominant copy number signature or the one or more copy number signature components are identified according to the method of any one of clauses 43 to 78.
   89. A method for monitoring cancer progression or recurrence in an individual, the method comprising:
identifying a first dominant copy number signature, or a first set of one or more copy number signature components, in a first copy number profile for a first sample obtained from an individual at a first time point according to the method of any one of clauses 43 to 78;
identifying a second dominant copy number signature, or a second set of one or more copy number signature components, in a second copy number profile for a second sample obtained from the individual at a second time point; and comparing the first dominant copy number signature, or the first set of one or more copy number signature components, to the second dominant copy number signature, or the second set of one or more copy number signature components, thereby monitoring the cancer progression or recurrence.
   90. The method of clause 85, wherein the second dominant copy number signature, or the second set of one or more copy number signature components, for the second sample is determined according to the method of any one of clauses 43 to 78.
   91. The method of clause 89 or clause 90, further comprising selecting an anti-cancer therapy for the individual in response to the cancer progression.
   92. The method of clause 89 or clause 90, further comprising administering an anti-cancer therapy to the individual in response to the cancer progression.
   93. The method of clause 89 or clause 90, further comprising adjusting an anti-cancer therapy for the individual in response to the cancer progression.
   94. The method of any one of clauses 91 to 93, further comprising adjusting a dosage of the anti-cancer therapy or selecting a different anti-cancer therapy in response to the cancer progression.
   95. The method of clause 94, further comprising administering the adjusted anti-cancer therapy to the individual.
   96. The method of any one of clauses 89 to 95, wherein the first time point is before the individual has been administered an anti-cancer therapy, and wherein the second time point is after the individual has been administered the anti-cancer therapy.
   97. The method of any one of clauses 89 to 96, wherein the individual has a cancer, is at risk of having a cancer, is being routine tested for cancer, or is suspected of having a cancer.
   98. The method of any one of clauses 89 to 97, wherein the cancer is a solid tumor.
   99. The method of any one of clauses 89 to 97, wherein the cancer is a hematological cancer.
   100. The method of any one of clauses 90 to 99, wherein the anti-cancer therapy comprises chemotherapy, radiation therapy, immunotherapy, a targeted therapy, or surgery.
   101. The method of any one of clauses 43 to 78, further comprising determining, identifying, or applying the dominant copy number signature, or the one or more copy number signature components, for the sample as a diagnostic value associated with the sample.
   102. The method of any one of clauses 43 to 78, further comprising generating a genomic profile for the individual based on the identification of a dominant copy number signature or of one or more copy number signature components in a copy number profile for the sample from the individual.
   103. The method of clause 102, wherein the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.
   104. The method of clause 102 or clause 103, wherein the genomic profile for the individual further comprises results from a nucleic acid sequencing-based test.
   105. The method of any one of clauses 102 to 104, further comprising selecting an anti-cancer therapy, administering an anti-cancer therapy, or applying an anti-cancer therapy to the individual based on the generated genomic profile.
   106. The method of any one of clauses 43 to 78, wherein the identification of a dominant copy number signature or of one or more copy number signature components in a copy number profile for the sample from the individual is used in making suggested treatment decisions for the subject.
   107. The method of any one of clauses 43 to 78, wherein the identification of a dominant copy number signature or of one or more copy number signature components in a copy number profile for the sample from the individual is used in applying or administering a treatment to the subject.
   108. A method for identifying an individual that may benefit from treatment with a poly (ADP-ribose) polymerase inhibitor (PARPi), comprising:
receiving, at one or more processors, targeted sequence read data for a sample derived from an individual diagnosed with or suspected of having cancer;
determining, using the one or more processors, a copy number profile having a plurality of copy number features for the individual based on the targeted sequence read data for the sample;
determining, using the one or more processors, a copy number feature value for each of the plurality of copy number features;
performing, using the one or more processors, an analysis of the copy number feature values for each of a plurality of copy number features using a first trained model to determine a probability that each of one or more copy number feature subgroups is represented in the copy number profile for the sample;
performing, using the one or more processors, an analysis of the probabilities determined for each of the one or more copy number feature subgroups using a second trained model to identify one or more copy number signatures represented in the copy number profile for the individual, and to determine one or more corresponding copy number signature scores;
comparing, using the one or more processors, the copy number signature scores for the one or more identified copy number signatures to one or more corresponding predetermined thresholds; and
identifying, based on the comparison, a dominant copy number signature for the individual, wherein the dominant copy number signature comprises a homologous recombination deficient (HRD) signature; or
identifying, based on the comparison, one or more copy number signatures that are components of the copy number profile for the individual, wherein the one or more copy number signatures comprise a homologous recombination deficient (HRD) signature;
   wherein identification of the HRD signature indicates that the individual may benefit from treatment with a PARPi.
   109. The method of clause 108, further comprising administering an effective amount of a PARPi to the individual.
   110. The method of clause 108 or clause 109, wherein the PARPi comprises olaparib, rucaparib, niraparib, talazoparib, or veliparib.
   111. The method of any one of clauses 108 to 110, wherein the cancer is ovarian cancer.
   112. The method of any one of clauses 108 to 110, wherein the cancer is prostate cancer.
   113. The method of any one of clauses 108 to 112, further comprising, prior to receiving the targeted sequence read data:
providing a plurality of nucleic acid molecules obtained from a sample from the individual;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules; and
sequencing, by a sequencer, the captured nucleic acid molecules to obtain a plurality of sequence reads that represent the captured nucleic acid molecules.
   114. The method of any one of clauses 108 to 113, wherein the sample lacks a loss-of-function mutation in BRCA1and/or BRCA2 gene(s).
   115. The method of clause 114, wherein the sample comprises biallelic loss-of-function mutations in one or more of BARD1, PALB2, RAD51D, and RAD51C gene(s).
   116. The method of clause 114, wherein the sample further lacks a loss-of-function mutation in ATM, BARD1, BRIP1, CDK12, CHEK1, CHEK2, FANCL, PALB2, RAD51B, RAD51C, RAD51D, and RAD54L gene(s).
   117. The method of any one of clauses 108 to 116, wherein the HRD signature comprises a plurality of low copy number segments.
   118. The method of any one of clauses 108 to 117, wherein the HRD signature comprises heterozygous deletion(s), high genome-wide loss of heterozygosity (gLOH-high), and/or high genomic instability score (GIS).
   119. The method of any one of clauses 111 and 113 to 118, wherein the cancer is ovarian or breast cancer, and wherein the HRD signature comprises heterozygous deletion(s), heterozygous duplication(s), and high genome-wide loss of heterozygosity (gLOH-high).
   120. The method of any one of clauses 112 and 113 to 118, wherein the cancer is prostate cancer, and wherein the HRD signature comprises heterozygous deletion(s) and copy neutral loss of heterozygosity.
   121. The method of any one of clauses 108 to 120, wherein the sample comprises a tissue biopsy sample, a liquid biopsy sample, or a normal control.
   122. The method of any one of clauses 108 to 120, wherein the sample is a liquid biopsy sample and comprises blood, plasma, cerebrospinal fluid, sputum, stool, urine, or saliva.
   123. The method of any one of clauses 108 to 120, wherein the sample is a liquid biopsy sample and comprises circulating tumor cells (CTCs).
   124. The method of any one of clauses 108 to 120, wherein the sample is a liquid biopsy sample and comprises cell-free DNA (cfDNA), circulating tumor DNA (ctDNA), or any combination thereof.
   125. A system comprising:
one or more processors; and
a memory communicatively coupled to the one or more processors and configured to store instructions that, when executed by the one or more processors, cause the system to:
   receive targeted sequence read data for a sample derived from an individual diagnosed with or suspected of having a disease;
   determine a copy number profile having a plurality of copy number features for the individual based on the targeted sequence read data for the sample;
   determine a copy number feature value for each of the plurality of copy number features;
   perform an analysis of the copy number feature values for each of a plurality of copy number features using a first trained model to determine a probability that each of one or more copy number feature subgroups is represented in the copy number profile for the sample;
   perform an analysis of the probabilities determined for each of the one or more copy number feature subgroups using a second trained model to identify one or more copy number signatures represented in the copy number profile for the individual, and to determine one or more corresponding copy number signature scores;
   compare the copy number signature scores for the one or more identified copy number signatures to one or more corresponding predetermined thresholds; and
   identify, based on the comparison, a dominant copy number signature for the individual; or
   identify, based on the comparison, one or more copy number signatures that are components of the copy number profile for the individual.
      126. The system of clause 125, wherein the copy number profile for the individual is determined based on off-target sequence reads in the targeted sequence read data for the sample.
      127. The system of clause 125 or clause 126, wherein the copy number profile for the individual is determined based on single nucleotide polymorphisms (SNPs) located in the targeted sequence read data for the sample.
      128. The system of clause 127, wherein the single nucleotide polymorphisms (SNPs) are located in on-target sequence read data.
      129. The system of any one of clauses 125 to 127, wherein the copy number profile for the individual is determined based on off-target sequence reads in the targeted sequence read data for the sample.
      130. The system of any one of clauses 125 to 129, wherein the copy number profile for the individual is determined based on a combination of on-target and off-target sequence reads in the targeted sequence read data for the sample.
      131. The system of clause 129 or clause 130, wherein the off-target sequence reads comprise an average coverage of less than 0.5×, 0.4×, 0.3×, 0.2×, or 0.1×.
      132. The system of any one of clauses 125 to 131, wherein the analysis to identify one or more copy number signatures represented in the copy number profile of the individual comprises an analysis of both the probabilities determined for each of the one or more copy number feature subgroups and a presence of one or more genomic features detected in the targeted sequence read data for the individual using the second trained model.
      133. The system of clause 132, wherein the one or more genomic features comprise one or more short variants, one or more indels, one or more biallelic loss-of-function variants, one or more rearrangements, or any combination thereof.
      134. The system of any one of clauses 125 to 133, wherein the plurality of copy number features comprises a number of breakpoints per 10 Mb of genomic sequence, a number of breakpoints per 25 Mb of genomic sequence, a number of breakpoints per 50 Mb of genomic sequence, a number of breakpoints per 100 Mb of genomic sequence, a number of breakpoints per chromosome arm for each chromosome, a magnitude of a copy number change between any two adjacent copy number segments; a length of each copy number segment; the copy number of segments; a count of contiguous oscillating copy number chains, or any combination thereof.
      135. The system of any one of clauses 125 to 134, wherein the first trained model comprises a trained statistical model.
      136. The system of any one of clauses 125 to 134, wherein the first trained model comprises a trained machine learning model.
      137. The system of any one of clauses 125 to 134, wherein the first trained model is updated based on the one or more copy number signatures identified in the copy number profile of the individual.
      138. The system of any one of clauses 125 to 137, wherein the first trained model comprises a latent class analysis model or a mixture model.
      139. The system of any one of clauses 125 to 138, wherein the second trained model comprises a non-negative matrix factorization (NMF) model or a machine learning model.
      140. The system of clause 139, wherein the second trained model comprises a machine learning model, and the machine learning model comprises a supervised, semi-supervised, or unsupervised machine learning model.
      141. The system of clause 140, wherein the second trained model comprises a supervised machine learning model, and the supervised machine learning model comprises an XGBoost model.
      142. The system of any one of clauses 125 to 141, wherein the one or more corresponding predetermined thresholds are determined based on an analysis of copy number signature scores for a cohort of individuals diagnosed with the disease.
      143. The system of any one of clauses 125 to 142, wherein the one or more corresponding predetermined thresholds are determined based on an analysis of copy number signature scores for a cohort of individuals comprising individuals diagnosed with a plurality of different diseases.
      144. The system of any one of clauses 125 to 143, wherein the one or more corresponding predetermined thresholds are determined based on an analysis of data for a cohort of individuals that comprises both copy number signature scores and clinical data.
      145. A non-transitory computer-readable storage medium storing one or more programs, the one or more programs comprising instructions, which when executed by one or more processors of a system, cause the system to:
receive targeted sequence read data for a sample derived from an individual diagnosed with or suspected of having a disease;
determine a copy number profile having a plurality of copy number features for the individual based on the targeted sequence read data for the sample;
determine a copy number feature value for each of a plurality of copy number features;
perform an analysis of the copy number feature values for each of a plurality of copy number features using a first trained model to determine a probability that each of one or more copy number feature subgroups is represented in the copy number profile for the sample;
perform an analysis of the probabilities determined for each of the one or more copy number feature subgroups using a second trained model to identify one or more copy number signatures represented in the copy number profile for the individual, and to determine one or more corresponding copy number signature scores;
compare the copy number signature scores for the one or more identified copy number signatures to one or more corresponding predetermined thresholds; and
identify, based on the comparison, a dominant copy number signature for the individual; or
identify, based on the comparison, one or more copy number signatures that are components of the copy number profile for the individual.
   146. The non-transitory computer-readable storage medium of clause 145, wherein the copy number profile for the individual is determined based on off-target sequence reads in the targeted sequence read data for the sample.
   147. The non-transitory computer-readable storage medium of clause 145 or clause 146, wherein the copy number profile for the individual is determined based on single nucleotide polymorphisms (SNPs) located in the targeted sequence read data for the sample.
   148. The non-transitory computer-readable storage medium of clause 147, wherein the single nucleotide polymorphisms (SNPs) are located in on-target sequence read data.
   149. The non-transitory computer-readable storage medium of any one of clauses 145 to 148, wherein the copy number profile for the individual is determined based on off-target sequence reads in the targeted sequence read data for the sample.
   150. The non-transitory computer-readable storage medium of any one of clauses 145 to 149, wherein the copy number profile for the individual is determined based on a combination of on-target and off-target sequence reads in the targeted sequence read data for the sample.
   151. The non-transitory computer-readable storage medium of clause 149 or clause 150, wherein the off-target sequence reads comprise an average coverage of less than 0.5×, 0.4×, 0.3×, 0.2×, or 0.1×.
   152. The non-transitory computer-readable storage medium of any one of clauses 145 to 151, wherein the analysis to identify one or more copy number signatures represented in the copy number profile of the individual comprises an analysis of both the probabilities determined for each of the one or more copy number feature subgroups and a presence of one or more genomic features detected in the targeted sequence read data for the individual using the second trained model.
   153. The non-transitory computer-readable storage medium of clause 152, wherein the one or more genomic features comprise one or more short variants, one or more indels, one or more biallelic loss-of-function variants, one or more rearrangements, or any combination thereof.
   154. The non-transitory computer-readable storage medium of any one of clauses 145 to 153, wherein the plurality of copy number features comprises a number of breakpoints per 10 Mb of genomic sequence, a number of breakpoints per 25 Mb of genomic sequence, a number of breakpoints per 50 Mb of genomic sequence, a number of breakpoints per 100 Mb of genomic sequence, a number of breakpoints per chromosome arm for each chromosome, a magnitude of a copy number change between any two adjacent copy number segments; a length of each copy number segment; the copy number of segments; a count of contiguous oscillating copy number chains, or any combination thereof.
   155. The non-transitory computer-readable storage medium of any one of clauses 145 to 154, wherein the first trained model comprises a trained statistical model.
   156. The non-transitory computer-readable storage medium of any one of clauses 145 to 154, wherein the first trained model comprises a trained machine learning model.
   157. The non-transitory computer-readable storage medium of any one of clauses 145 to 154, wherein the first trained model is updated based on the one or more copy number signatures identified in the copy number profile of the individual.
   158. The non-transitory computer-readable storage medium of any one of clauses 145 to 157, wherein the first trained model comprises a latent class analysis model or a mixture model.
   159. The non-transitory computer-readable storage medium of any one of clauses 145 to 158, wherein the second trained model comprises a non-negative matrix factorization (NMF) model or a machine learning model.
   160. The non-transitory computer-readable storage medium of clause 159, wherein the second trained model comprises a machine learning model, and the machine learning model comprises a supervised, semi-supervised, or unsupervised machine learning model.
   161. The non-transitory computer-readable storage medium of clause 160, wherein the second trained model comprises a supervised machine learning model, and the supervised machine learning model comprises an XGBoost model.
   162. The non-transitory computer-readable storage medium of any one of clauses 145 to 161, wherein the one or more corresponding predetermined thresholds are determined based on an analysis of copy number signature scores for a cohort of individuals diagnosed with the disease.
   163. The non-transitory computer-readable storage medium of any one of clauses 145 to 162, wherein the one or more corresponding predetermined thresholds are determined based on an analysis of copy number signature scores for a cohort of individuals comprising individuals diagnosed with a plurality of different diseases.
   164. The non-transitory computer-readable storage medium of any one of clauses 145 to 163, wherein the one or more corresponding predetermined thresholds are determined based on an analysis of data for a cohort of individuals that comprises both copy number signature scores and clinical data.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method, comprising:
providing a plurality of nucleic acid molecules obtained from a sample from an individual diagnosed with or suspected of having a disease;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, targeted sequence read data for the sample derived from the individual;
determining, using the one or more processors, a copy number profile having a plurality of copy number features for the individual based on the targeted sequence read data for the sample;
determining, using the one or more processors, a copy number feature value for each of the plurality of copy number features;
performing, using the one or more processors, an analysis of the copy number feature values for each of a plurality of copy number features using a first trained model to determine a probability that each of one or more copy number feature subgroups is represented in the copy number profile for the sample;
performing, using the one or more processors, an analysis of the probabilities determined for each of the one or more copy number feature subgroups using a second trained model to identify one or more copy number signatures represented in the copy number profile for the individual, and to determine one or more corresponding copy number signature scores;
comparing, using the one or more processors, the copy number signature scores for the one or more identified copy number signatures to one or more corresponding predetermined thresholds; and
identifying, based on the comparison, a dominant copy number signature for the individual; or
identifying, based on the comparison, one or more copy number signatures that are components of the copy number profile for the individual.

2. The method of claim 1, further comprising: (i) confirming a diagnosis of the disease for the individual, (ii) predicting a treatment outcome for the individual, (iii) selecting a treatment for the individual, (iv) adjusting a treatment dosage for the individual, (v) selecting the individual for inclusion in a clinical trial, (vi) identifying the individual as a candidate for chemotherapy, radiation therapy, or surgery, and/or (vii) identifying the individual as a candidate for hospice care based on the dominant copy number signature or the one or more copy number signatures identified for the individual.

3. (canceled)

4. (canceled)

5. (canceled)

6. (canceled)

7. (canceled)

8. (canceled)

9. The method of claim 1, wherein the copy number profile for the individual is determined based on single nucleotide polymorphisms (SNPs) located in the targeted sequence read data for the sample.

10. The method of claim 1, wherein the copy number profile for the individual is determined based on off-target sequence reads in the targeted sequence read data for the sample.

11. The method of claim 1, wherein the copy number profile for the individual is determined based on a combination of on-target and off-target sequence reads in the targeted sequence read data for the sample.

12. (canceled)

13. The method of claim 1, wherein the analysis to identify one or more copy number signatures represented in the copy number profile of the individual comprises an analysis of both the probabilities determined for each of the one or more copy number feature subgroups and a presence of one or more genomic features detected in the targeted sequence read data for the individual using the second trained model.

14. The method of claim 1, wherein the plurality of copy number features comprises a number of breakpoints per 10 Mb of genomic sequence, a number of breakpoints per 25 Mb of genomic sequence, a number of breakpoints per 50 Mb of genomic sequence, a number of breakpoints per 100 Mb of genomic sequence, a number of breakpoints per chromosome arm for each chromosome, a magnitude of a copy number change between any two adjacent copy number segments; a length of each copy number segment; the copy number of segments; a count of contiguous oscillating copy number chains, or any combination thereof.

15. The method of claim 1, wherein the first trained model comprises a trained statistical model or a trained machine learning model.

16. The method of claim 1, wherein the first trained model is trained on copy number feature values calculated for each of the plurality of copy number features based on copy number profiles for a cohort of individuals diagnosed with a cancer.

17. The method of claim 1, wherein the second trained model comprises a non-negative matrix factorization (NMF) model or a machine learning model.

18. The method of claim 1, wherein the second trained model is trained on copy number feature subgroups identified in data for a cohort of individuals diagnosed with a cancer.

19. The method of claim 1, wherein the second trained model is trained to identify copy number signatures based on the probabilities determined for each of the one or more copy number feature subgroups and based on the presence of one or more additional genomic features identified in the targeted sequence read data.

20. The method of claim 1, wherein the one or more corresponding predetermined thresholds are determined based on an analysis of copy number signature scores for a cohort of individuals diagnosed with a cancer.

21. The method of claim 1, wherein the one or more corresponding predetermined thresholds are determined based on an analysis of data for a cohort of individuals that comprises both copy number signature scores and clinical data.

22. A method for identifying an individual that may benefit from treatment with a poly (ADP-ribose) polymerase inhibitor (PARPi), comprising:
receiving, at one or more processors, targeted sequence read data for a sample derived from an individual diagnosed with or suspected of having cancer;
determining, using the one or more processors, a copy number profile having a plurality of copy number features for the individual based on the targeted sequence read data for the sample;
determining, using the one or more processors, a copy number feature value for each of the plurality of copy number features;
performing, using the one or more processors, an analysis of the copy number feature values for each of a plurality of copy number features using a first trained model to determine a probability that each of one or more copy number feature subgroups is represented in the copy number profile for the sample;
performing, using the one or more processors, an analysis of the probabilities determined for each of the one or more copy number feature subgroups using a second trained model to identify one or more copy number signatures represented in the copy number profile for the individual, and to determine one or more corresponding copy number signature scores;
comparing, using the one or more processors, the copy number signature scores for the one or more identified copy number signatures to one or more corresponding predetermined thresholds; and
identifying, based on the comparison, a dominant copy number signature for the individual, wherein the dominant copy number signature comprises a homologous recombination deficient (HRD) signature; or
identifying, based on the comparison, one or more copy number signatures that are components of the copy number profile for the individual, wherein the one or more copy number signatures comprise a homologous recombination deficient (HRD) signature;
wherein identification of the HRD signature indicates that the individual may benefit from treatment with a PARPi.

23. The method of claim 22, further comprising administering an effective amount of a PARPi to the individual.

24. The method of claim 22, wherein the PARPi comprises olaparib, rucaparib, niraparib, talazoparib, or veliparib.

25. The method of claim 22, wherein the cancer is ovarian cancer or prostate cancer.

26. The method of claim 22, wherein the sample lacks a loss-of-function mutation in BRCA1 and/or BRCA2 gene(s).

27. (canceled)

28. (canceled)

29. The method of claim 22, wherein the HRD signature comprises a plurality of low copy number segments.

30. (canceled)

31. (canceled)

32. (canceled)

33. (canceled)
